(19) United States
(12) Patent Application Publication
(10) Pub. No.: US 2006/0105323 A1
(43) Pub. Date: May 18, 2006

(54) Multi-reporter gene model for toxicological screening

(75) Inventors: Christopher Bruce Alexander Whitelaw, Roslin (GB); Anthony John Clark, Roslin (GB); Helen Clark, Roslin (GB); Wolf Charles Roland, Dundee Technopole (GB)

Correspondence Address: WILMER CUTLER PICKERING HALE AND DORR LLP, 60 STATE STREET, BOSTON, MA, 02109, US

(73) Assignee: Roslin Institute (Edinburgh) Biosciences Ltd., Dundee Technopole (GB)

(21) Appl. No.: 10/522,356

(22) PCT Filed: Jul. 25, 2003
(86) PCT No.: PCT/GB03/03192

§ 371 Date: Jan. 26, 2005

(30) Foreign Application Priority Data
Jul. 26, 2002 (GB) 0217402.7

(51) Int. Cl. C12Q 1/70; C12Q 1/68

(52) U.S. Cl. 435/5; 435/6

(57) ABSTRACT

A system for the detection of gene activation events is provided which comprises a nucleic acid construct encoding a protein of the lipocalin protein family and a peptide tag in which the expression of the construct in a cell or in the cells of a transgenic animal demonstrates the activation of a gene or genes of interest, in which the protein expressed is secreted from the cell and in which detection of the peptide tag indicates expression of the construct.

The present invention relates to a non-invasive reporter gene system for the detection of gene activation events related to altered metabolic status in vivo or in vitro for use in toxicological screening.

Genes encode proteins. It is estimated that there at least 3×10⁴ genes in the vertebrate genome but for a given cell only a subset of the total number of genes is active, with the subset differing between cells of different types and between different stages of development and differentiation (Cho & Campbell Trends Genet. 16 409-415 (2000); Velculescu et al Trends Genet. 16 423-425 (2000)). The DNA regulatory elements associated with each gene governs the decision as to which genes are active and which are not. Although comprising a number of defined elements these DNA sequences are collectively termed promoters (Tjian & Maniatis Cell 77 5-8 (1994); Bonifer, Trends Genet. 16 310-315 (2000); Martin, Trends Genet 17 444-448 (2001)).

Gene activation occurs primarily at the transcriptional level. Transcriptional activity of a gene may be measured by a variety of approaches including RNA polymerase activity, mRNA abundance or protein production (Takano et al., 2002). These approaches are limited in that they require development of an assay suitable to each individual mRNA or protein product. To facilitate comparison of different promoters, rather than assaying individual gene products, reporter genes are often used (Sun et al Gene Ther. 8 1572-1579 (2001); Franco et al Eur. J. Morphol. 39 169-191 (2001); Hadjantonakis & Nagy, Histochem. Cell. Biol. 115 49-58 (2001); Gorman Mol. Cell. Biol. 2 1044-1051 (1982); Barash and Reichenstein, 2002; Zhang et al., 2001.).

The product (mRNA or protein) of a reporter gene allows an assessment of the transcriptional activity of a particular gene and can be used to distinguish cells, tissues or organisms in which the event has occurred from those in which it has not. On the whole reporter genes are foreign to the host cell or organism, allowing their activity to be easily distinguished from the activity of endogenous genes. Alternatively the reporter may be marked or tagged so as to make it distinct from host genes.

Reporter genes are linked to the test promoter, enabling activity of the promoter gene to be determined by detecting the presence of the reporter gene product. Therefore, the main prerequisite for a reporter gene product is that it is easy to detect and quantify. In some cases, but not all, the reporter gene has enzymatic activity that catalyses the conversion of a substrate into a measurable product.

A classical example is the bacterial chloramphenicol acetyl transferase (CAT) gene. CAT activity can be measured in cell extracts as conversion of added non-acetylated chloramphenicol to the acetylated form of chloramphenicol by chromatography (Gorman Mol. Cell. Biol. 2 1044-1051 (1982)). Similar strategies enable the use of the firefly luciferase gene as a reporter. In this instance it is the light produced by bioluminescence of the luciferin substrate that is measured.

Some reporters also benefit from the visual detection assays that allow in situ analysis of reporter activity. A frequently used example would be β-galactosidase (Lac Z), where the addition of an artificial substrate, X-gal, enables reporter activity to be detected by the appearance of blue colouration in the sample. As it is accumulative it effectively provides an historical record of its induction. This is particularly useful for measuring transient responses where a promoter is activated for only a short time before being rapidly inactivated. This reporter has been successfully used both in cultured cells and in vivo (Campbell et al J. Cell. Biol. 109 2619-2625 (1996)), though its suitability for in vivo use has been questioned in some reports (Sanchez-Ramnos et al Cell Transplant. 9 657-667 (2000); Montoliu et al Transgenic Res. 9 237-239 (2000); Cohen-Tannoudji et al Transgenic Res. 9 233-235 (2000)). It has been demonstrated that Lac Z in combination with fluorescent substrates can enable the sorting of cells that express the reporter by use of a fluorescence-activated cell sorter (FACS) (Fiering et al Cytometry 12 291-301 (1991)).

In other systems, the reporter product itself is directly detected, removing the need for a substrate. Green fluorescent protein has become on of the most commonly used examples of this category of reporter (Ikawa et al Curr. Top. Dev. Biol. 44 1-20 (1997)). This autofluorescing protein was derived from the bioluminescent jellyfish Aequoria victoria. Several colour spectral variants of this reporter have been developed (Hadjantonakis & Nagy, Histochem. Cell. Biol. 115 49-58 (2001)).

Recently reporter systems based on energy emission systems have been developed. These include single photon emission computed tomography (SPECT) and positron emission tomography (PET) though these require the introduction of a radiolabelled isotope probe in to the host cell or animal that is then modified by the target reporter gene. For example the PET system measures reporter sequestering of the positron emitting probe (Sun et al Gene Ther. 8 1572-1579 (2001)). These are summarised as follows:

Established reporter

| Enzymatic | Light based |
|---|---|
| alkaline phosphatase | Green fluorescent protein |
| Beta galactosidase | dsRed |
| Thymidine kinase | Luciferase |
| Neomycin resistance | |
| Chloramphenicol acetyl transferase | |
| Growth hormone | |

Many tried and tested reporter systems have been developed but nevertheless share certain limitations. Those based on prokaryote genes often suffer poor expression in transgenic mammals (Montoliu et al Transgenic Res. 9 237-238 (2000); Cohen-Tannoudji et al Transgenic Res. 9 233-235 (2000)). Furthermore the presence of prokaryote DNA sequences has been implicated in the suppression of expression from adjacent eukaryote transgenes as have the presence of intronless, cDNA based eukaryote gene sequences (Clark et al., 1997).

Most of the current reporters, whilst useful for monitoring expression under certain circumstances, have certain limitations. Many accumulate in cells and are not useful for monitoring changes in promoter activation over time. Perhaps more importantly detection of expression necessitates the fixing of cultured cells or the sacrifice of transgenic animals, thus limiting reporters to invasive detection strategies. There are a few exceptions and these include the use of growth hormone (Bchini et al Endocrinology 128 539-546 (1991)). However its high biological activity effectively limit its widespread applicability. Another enzyme that has been used in vivo is a secreted version of alkaline phosphatase (SEAP) (Nilsson et al Cancer Chemother. Pharmacol. 49 93-100 (2002); Durocher Nucl. Acids. Res. 30 E9 (2002)) though again, the potential biological effects resulting from its heterologous expression remain untested. GFP has been detected in whole animals and though possessing relatively low biological activity its use has so far been limited to neonatal and nude mice in which both internal tissue and dermal fluorescence are more readily observed. In addition there has been a report that GFP is cytotoxic (Liu et al Biochem. Biophys. Res. Comm. 260 712-717 (1999)). Although reporter systems based on tomography allow monitoring of reporter expression in internal tissues they require addition of exogenously added substrates that could potentially confound results by influencing expression of the reporter. Additionally they can lack the sensitivity required for quantitative analysis of reporter expression.

There is therefore a need for a reporter system that overcomes some or all of these limitations. Primarily it should be non-invasive inasmuch as its detection does not involve addition of an external substrate or sacrifice of transgenic animals. This would also ideally stipulate that the reporter be secreted (in vitro and in vivo) or excreted (in vivo). Secondly it should be biologically neutral with regard to the test expression system so that no phenotypic effects either confound readout from the system or affect the health of the transgenic animal. Thirdly a family of reporters sharing similar and therefore predictable characteristics allowing comparison between reporters is required. This may be achieved if members share a common structure or backbone.

A system satisfying these requirements has now been found. The members of the lipocalin protein family fulfil the necessary characteristics for a non-invasive reporter.

According to a first aspect of the invention, there is provided a nucleic acid construct comprising (i) a nucleic acid sequence encoding a member of the lipocalin protein family, and (ii) a nucleic acid sequence encoding a peptide sequence of from 5 to 250 amino acid residues

The lipocalins are a diverse family of small molecule transporter proteins that share a common conserved gene structure (Flower et al Biochim. Biophys Acta 1482 9-24 (2000)). Members of this family are small in size with the majority falling into the 18-25 kD range. Some are naturally secreted, e.g. ovine betalactoglobulin (BLG) (accession No. X12817), or excreted e.g. murine major urinary protein (MUP) (e.g. accession No. NM 031188) and rat α-2-urinary globulin (α-2u) (accession number M27434). Lipocalin reporters will preferably be either MUP, BLG or α-2u but could be chosen from the following list of other lipocalin family members shown in Table 1:

TABLE 1

| | Subunit | | | | | | |
|---|---|---|---|---|---|---|---|
| | molecular | | No. | Oligomeric | | No. | |
| Protein | mass | pI | residues | State | Glycosyln. | S═S | Abbr./ref |
| Kernel lipocalins | | | | | | | |
| Retinol-binding | 21.0 | 5.5 | 183 | Monomer | − | 3 | RBP (1), |
| protein | | | | | | | (2) |
| Purpurin | 20.0 | | 175 | | | | PURP (3) |
| Retinoic acid- | 18.5 | 5.2 | 166 | Monomer | − | 1 | RABP (4) |
| binding protein | | | | | | | |
| α₂ᵤ-Globulin | 18.7 | 5.7-6.7 | 162 | Dimer | − | 1 | A2U (5)-(7) |
| Major urinary | 17.8 | 5.5-5.7 | 161 | Dimer | − | 1 | MUP (8)-(10) |
| protein | | | | | | | |
| Bilin-binding | 19.6 | | 173 | Tetramer | − | 2 | BBP (11) |
| protein | | | | | | | |
| α- | 350.0 | 4.3-4.7 | 174/181 | Octamer of | − | 2/2 | (12) (13) |
| Crustacyanin | | | | heterodimers | | | |
| Pregnancy | 56.0 | | 162 | Homodimer | + | | PP14 (15) |
| protein 14 | | | | | | | |
| β- | 18.0 | 5.2 | 162 | Dimer/ | − | 2 | Big (16)-(18) |
| Lactoglobulin | | | | monomer | | | |
| α₁- | 31.0 | 4.3-4.8 | 188 | Monomer + complexes | + | 1 | A1M (19) |
| Microglobulin | | | | | | | |
| C8γ | 22.0 | | 182 | Part of complex | − | 1 | C8γ (20) |
| Apolipoprotein D | 29.0-32.0 | 4.7-5.2 | 169 | Dimer + complexes | + | 2 | ApoD |
| | | | | | | | (21)-(23) |
| Lazarillo | 45.0 | | 183 | Monomer | + | + | LAZ (24) |
| Prostaglandin | 27.0 | 4.6 | 168 | Monomer | + | 1 | PGDS |
| D synthase | | | | | | | (25) |
| Quiescence- | 21.0 | 6.3 | 158 | | | 1 | QSP (26)-(28) |
| specific protein | | | | | | | |
| Neutrophil | 25.0 | | 179 | Monomer/ | | | NGAL |
| lipocalin | | | | Dimer + complexes | | | (29)-(32) |
| Choroid plexus | 20.0 | | 183 | Monomer | − | | (33) |
| protein | | | | | | | |
| Outlier | | | | | | | |
| lipocalins | | | | | | | |
| Odorant- | 37.0-40.0 | 4.7 | 159 | Dimer | | 0 | OBP (34)-(36) |
| binding protein | | | | | | | |
| von Ebner's- | 18.0 | 4.8-5.2 | 170 | Dimer | | 1 | VEGP |
| gland protein | | | | | | | (37)-(40) |
| α₁-Acid | 40.0 | 3.2 | 183 | Monomer | + | 2 | AGP |
| glycoprotein | | | | | | | (41)(42) |
| Probasin | 20.0 | 11.5 | 160 | | | | PBAS (43) |
| Aphrodisin | 17.0 | | 151 | | + | 2 | (44) |

“Glycosyln”. = glycosylation
“No. S═S” = no. of disulphides
References:
(1) Cogan et al Eur J. Biochem 65, 71-78 (1976).
(2) Hase et al J. Biochem 79 373-380 (1976)
(3) Berman et al Cell 51 135-142 (1987)
(4) Newcomer, M. E. & Ong, D. E., J. Biol. Chem. 265 12876-12879 (1990)
(5) Borghoff et al Ann. Rev. Pharmacol. Toxicol. 30 349-367 (1990)
(6) Cavaggioni et al Comp. Biochem. Physiol. 96B 513-520 (1990)
(7) Borghoff et al Toxicol. Appl. Pharmacol. 107 228-238 (1991)
(8) Shaw et al Cell 32 755-761 (1983)
(9) Cavaggioni et al Comp. Biochem. Physiol. 96B 513-520 (1990)
(10) Borghoff et al Toxicol. Appl. Pharmacol. 107 228-238 (1991)
(11) Riley et al J. Biol. Chem. 259 13159-13165 (1984)
(12) Britton et al in “Carotenoid Chemistry and Biochemistry”, eds. Britton, G. & Goodwin, T. W., 237-253, Pergamon Press, Oxford (1982)
(13) Zagalsky et al Comp. Biochem. Physiol. 97B 1-18 (1990)
(14) Morrow et al Am. J. Pathol. 145 1485-1495 (1995)
(15) Hambling et al in “Advanced Dairy Chemistry”, volume 1, ed. Fox, P. F.,, 140-190, Elsevier Applied Science London (1992)
(16) Dodin et al Eur. J. Biochem. 193 697-700 (1990)
(17) Dufour et al FEBS Lett 277 223-226 (1991)
(18) Escribano et al Biochem. Biophys. Res. Comm. 155 1424-1429 (1988)
(19) Haefliger et al Mol. Immunol. 28 123-131 (1991)
(20) Balbin et al Biochem. J. 271 803-807 (1990)
(21) Peitsch, M. C. & Boguski, M. S., New Biol. 2 197-206 (1990)
(22) Morais Cabral et al FEBS Lett 366 53-56 (1995)
(23) Ganfornia et al Development 121 123-134 (1995)
(24) Urade et al J. Biol. Chem. 264 1041-1045 (1988)
(25) Cancedda et al J. Cell Biol. 107 2455-2463 (1990)
(26) Cancedda et al Biochem. Biophys. Res. Comm. 168 933-938 (1990)
(27) Nakano, T. & Graf, T. Oncogene 7 527-534 (1992)
(28) Hraba-Renevey et al Oncogene 4 601-608 (1989)
(29) Meheus et al J. Immunol. 152 1535-1547 (1993)
(30) Liu, Q., & Nilsen-Hamilton, M. J. Biol. Chem. 270 22565-22570 (1995)
(31) Kasik, J. W. & Rice, E. J. Am. J. Obstet. Gynecol. 173 613-617 (1995)
(32) Achen et al J. Biol. Chem. 267 23170-23174 (1992)
(33) Snyder et al J. Biol. Chem. 263 13971-13974 (1988)
(34) Lee et al Science 235 1053-1056 (1987)
(35) Cavaggioni et al FEBS Lett 212 225-228 (1987)
(36) Kock et al Physiol. Behav. 56 1173-1177 (1994)
(37) Schmale et al Ciba Found. Symp. 179 167-185 (1993)
(38) Redl et al J. Biol. Chem. 267 20282-20827 (1992)
(39) Glasgow et al Curr. Eye Res. 14 363-372 (1995)
(40) Kremer et al Pharmacol. Rev. 40 1-40 (1988)
(41) Arnaud et al Methods Enzymol. 163 418-431 (1988)
(42) Matuo et al Biochem. Biophys. Res. Comm. 118 467-473 (1984)
(43) Henzel et al J. Biol. Chem. 263 16682-16687 (1988)
(44) Magert et al Proc. Nat'l Acad. Sci. USA 92 2091-2095 (1995)

### REFERENCES

(1) Cogan et al Eur J. Biochem 65, 71-78 (1976).
(2) Hase et al J. Biochem 79 373-380 (1976)
(3) Berman et al Cell 51 135-142 (1987)
(4) Newcomer, M. E. & Ong, D. E., J. Biol Chem. 265 12876-12879 (1990)
(5) Borghoff et al Ann. Rev. Pharmacol. Toxicol. 30 349-367 (1990)
(6) Cavaggioni et al Comp. Biochem. Physiol. 96B 513-520 (1990)
(7) Borghoff et al Toxicol. Appl. Pharmacol. 107 228-238 (1991)
(8) Shaw et al Cell 32 755-761 (1983)
(9) Cavaggioni et al Comp. Biochem Physiol 96B 513-520 (1990)
(10) Borghoff et al Toxicol. Appl. Pharmacol. 107 228-238 (1991)
(11) Riley et al J. Biol. Chem. 259 13159-13165 (1984)
(12) Briton et al in “Carotenoid Chemistry and Biochemistry”, eds. Britton, G. & Goodwin, T. W., 237-253, Pergamon Press, Oxford (1982)
(13) Zagalasky et al Comp. Biochem. Physiol. 97B 1-18 (1982)
(14) Morrow et al Am. J. Pathol. 145 1485-1495 (1995)
(15) Hambling et al in “Advanced Dairy Chemistry”, volume 1, ed. Fox, P. F.,, 140-190, Elsevier Applied Science London (1992)
(16) Dodin et al Eur. J. Biochem. 193 697-700 (1990)
(17) Dufour et al FEBS Lett 277 223-226 (1991)
(18) Escribano et al Biochem. Biophys. Res. Comm. 155 1424-1429 (1998)
(19) Haefliger et al Mol. Immunol. 28 123-131 (1991)
(20) Balbin et al Biochem. J. 271 803-807 (1990)
(21) Peitsch, M. C. & Boguski, M. S., New Biol. 2 197-206 (1990)
(22) Morais Cabral et al FEBS Lett 366 53-56 (1995)
(23) Ganfornia et al Development 121 123-134 (1995)
(24) Urade et al J Biol. Chem. 264 1041-1045 (1998)
(25) Cancedda et al J. Cell Biol. 107 2455-2463 (1990)
(26) Cancedda et al Biochem. Biophys. Res. Comm. 168 933-938 (1990)
(27) Nakano, T. & Graf, T. Oncogene 7 527-534 (1992)
(28) Hraba-Renevey et al Oncogene 4 601-608 (1989)
(29) Meheus et al J. Immunol. 151 1535-1547 (1993)
(30) Liu, Q., & Nilsen-Hamilton, M. J. Biol. Chem 270 22565-22570 (1995)
(31) Kasid, J. W. & Rice, E. J. Am. J. Obstet. Gynecol. 173 613-617 (1995)
(32) Achen et al J. Biol. Chem. 267 23170-23174 (1992)
(33) Snyder et al J. Biol. Chem. 263 13971-13974 (1988)
(34) Lee et al Science 235 1053-1056 (1987)
(35) Cavaggioni et al FEBS Lett 212 225-228 (1987)
(36) Kock et al Physiol. Behav. 56 1173-1177 (1994)
(37) Schmale et al Ciba Found Symp. 179 167-185 (1993)
(38) Redl et al J. Biol. Chem. 20282-20827 (1992)
(39) Glasgow et al Curr. Eye Res. 14 363-372 (1995)
(40) Kremer et al Pharmacol. Rev. 40 1-40 (1998)
(41) Arnaus et al Methods Enzymol. 163 418-431 (1998)
(42) Matuo et al Biochem. Biophys. Res. Comm. 118 467-473 (1984)
(43) Henzel et al J. Biol. Chem. 263 16682-16687 (1988)
(44) Magert et al Proc. Nat'l Acad. Sci. USA 92 2091-2095 (1995)

The nucleic acid sequences of the present invention also include sequences that are homologous or complementary to those referred to above. The percent identity of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA (1990) 87:2264-2268, modified as in Karlin and Altschul (1993) Proc. Natl. Acad. Sci. USA 90:5873-5877. The NBLAST and XBLAST programs of Altschul et al, J. Mol. Biol. (1990) 215:403-410 have incorporated such an algorithm. BLAST nucleotide searches can be performed with the NBLAST program, score=100, wordlength=12 to obtain nucleotide sequences homologous to a nucleic acid molecules of the invention. To obtain gapped alignments for comparison purposes, Gapped BLAST can be utilised as described in Altschul et al, Nucleic Acids Res. (1997) 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., NBLAST) can be used. See www.ncbi.nlm.nih.gov.

Another example of a mathematical algorithm utilised for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. Appl. Biosci. (1994) 10:3-5; and FASTA described in Pearson and Lipman Proc. Natl. Acad. Sci. USA (1988) 85:2444-8. Within FASTA, ktup is a control option that sets the sensitivity and speed of the search.

A nucleic acid sequence which is complementary to a nucleic acid sequence of the present invention is a sequence which hybridises to such a sequence under stringent conditions, or a nucleic acid sequence which is homologous to or would hybridise under stringent conditions to such a sequence but for the degeneracy of the genetic code, or an oligonucleotide sequence specific for any such sequence. The nucleic acid sequences include oligonucleotides composed of nucleotides and also those composed of peptide nucleic acids. Where the nucleic sequence is based on a fragment of the sequences of the invention, the fragment may be at least any ten consecutive nucleotides from the gene, or for example an oligonucleotide composed of from 20, 30, 40, or 50 nucleotides.

Stringent conditions of hybridisation may be characterised by low salt concentrations or high temperature conditions. For example, highly stringent conditions can be defined as being hybridisation to DNA bound to a solid support in 0.5M NaHPO₄, 7% sodium dodecyl sulfate (SDS), 1 mM EDTA at 65° C., and washing in 0.1×SSC/0.1% SDS at 68° C. (Ausubel et al eds. “Current Protocols in Molecular Biology” 1, page 2.10.3, published by Green Publishing Associates, Inc. and John Wiley & Sons, Inc., New York, (1989)). In some circumstances less stringent conditions may be required. As used in the present application, moderately stringent conditions can be defined as comprising washing in 0.2×SSC/0.1% SDS at 42° C. (Ausubel et al (1989) supra). Hybridisation can also be made more stringent by the addition of increasing amounts of formamide to destabilise the hybrid nucleic acid duplex. Thus particular hybridisation conditions can readily be manipulated, and will generally be selected according to the desired results. In general, convenient hybridisation temperatures in the presence of 50% formamide are 42° C. for a probe which is 95 to 100% homologous to the target DNA, 37° C. for 90 to 95% homology, and 32° C. for 70 to 90% homology.

Examples of preferred nucleic acid sequences for use in according to the various aspects of the present invention are the sequences of the invention are disclosed herein. Complementary or homologous sequences may be 75%, 80%, 85%, 90%, 95%, 99% similar to such sequences.

With the addition of peptide tags to a chosen lipocalin reporter there is provided a useful sub-family of reporter proteins. Essentially it allows generation of a large number of reporters from a single lipocalin where that lipocalin acts as the carrier for a range of peptides that can be clearly differentiated from one another by a range or biological or physical assay techniques. For example it has been demonstrated that a casein kinase recognition sequence engineered in exon 3 of the ovine betalactoglobulin (BLG) gene resulted in expression of a novel form of BLG containing an active kinase substrate in one of the surface loops of the protein in transgenic mice (McClenaghan et al Protein Eng. 12 259-264 (1999)).

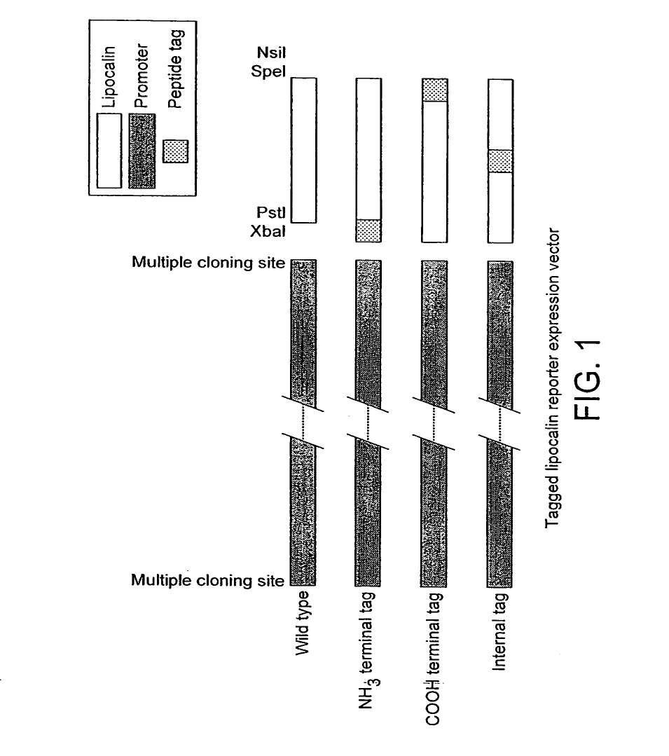

The position of the peptide tag may be at the amino terminal or carboxy terminal or inserted internally with respect to the amino acid sequence of the reporter. All three examples are represented in FIG. 1.

The peptide tag can be a sequence consisting of between 5 to 250 amino acids. Suitably, in the ranges of from, 5 to 50, 10 to 60, 20 to 70, 30 to 80, 40 to 90, and so on. In some embodiments of the invention peptides may be required to consist of a greater number of amino acids than 250 residues.

In a preferred embodiment of the invention the peptide tag may be an epitope, that is a defined amino acid sequence from a protein with a fully characterised cognate antibody. The skilled person can select such epitopes based on sequences identified as possessing antigenic properties. In certain embodiments of the invention the epitope tag may be the amino acid sequence below from the c-myc oncogene (Evans et al Mol. Cell. Biol. 5 3610-3616 (1985)):

-Glu-Gln-Lys-Leu-Ile-Ser-Glu-Glu-Asp-Leu-

(EQKLISEEDL)

or it may be the amino acid sequence from the simian virus V5 protein (Southern et al J. Gen. Virol. 72 1551-1557 (1991)), shown below:

-Gly-Lys-Pro-Ile-Pro-Asn-Pro-Leu-Leu-Gly-Leu-Asp-
Ser-Thr-

(GKPIPNPLLGLDST)

In certain embodiments of the invention, the epitope may be selected from but not limited to the c-myc and V5 proteins.

Other alternative epitopes may include, but are not limited to:

| | |
|---|---|
| Haemaglutinin | (YPYDVPDYA) |
| Clone100 | (NVRFSTIVRRRA) |
| rab11a | (KQMSDRRENDMSPS) |
| DOB | (SGNEVSRAVLLPQSC) |
| SG11 | (SSLSYTNPAVAATSANL) |
| erbB4 | (RSTLQHPDYLQEYST) |
| ARF | (VSTLLRWERFPGHRQA) |
| RYK | (KFQQLVQCLTEFHAALGAYV) |
| WILPEP1 | (QEQCQEVWRKRVISAFLKSP) |
| HAF10 | (RLSDKTGPVAQEKS) |

Preferably the epitope tag is recognised by its cognate antibody irrespective of whether it is located at the amino terminal, carboxy terminal or in an internal domain of the reporter protein.

In another embodiment of the invention the peptide tag may possess enzymatic activity that converts a substrate to a form that is readily detectable by an assay. For example a kinase activity specifying phosphorylation of another protein or peptide substrate that could be added to the secreted or excreted analyte along with a phosphate group donor. Detection could be achieved using an immunological assay based on detection by an antibody specifically recognising the phosphorylated version of the tagged reporter protein. Alternatively the use of phosphate radiolabelled with an isotope of phosphorous such as ³²P or ³³P. Other enzymic modifications include for example acetylation, sulphation and glycosylation. Another possibility is peptide tag that is an enzyme, that is the construct comprises a nucleic acid sequence encoding an enzyme, or a nucleic acid sequence encoding a catalytic sequence thereof, such as Glutathoine-S-transferase (GST) where enzyme activity can be detected by means of an activity assay or by antibody reactivity.

Suitably, the nucleic acid sequence encoding the member of the lipocalin protein family is contiguous with the nucleic acid sequence encoding the peptide sequence. However, a linker nucleic acid sequence may be inserted between these two sequences that encodes a short number of amino acids.

The nucleic acid construct may additionally comprise a promoter element upstream of the nucleic acid encoding the member of the lipocalin protein family. The promoter element may be an inducible promoter, preferably a stress inducible promoter.

It is also within the scope of the present invention for the nucleic acid construct to include more than one detectable peptide label. Such as for example, a peptide antigen and an enzyme (or an active catalytic site thereof). One possible combination is the peptide epitope c-myc and the enzyme GST.

Other embodiments of this aspect could include, for example site of interaction with protein other than antibody e.g. lectin binding site, or modification of tag by e.g. addition of amino acid multimer such as polylysine; or incorporation of a fluorochrome.

The peptide sequence may be as described above but it also extends to peptides and polypeptides that are substantially homologous thereto. The term “polypeptide” includes both peptide and protein, unless the context specifies otherwise.

Such peptides include analogues, homologues, orthologues, isoforms, derivatives, fusion proteins and proteins with a similar structure or are a related polypeptide as herein defined.

The term “analogue” as used herein refers to a peptide that possesses a similar or identical function as a peptide coded for by a nucleic acid sequence of the invention but need not necessarily comprise an amino acid sequence that is similar or identical to an amino acid sequence of the invention, or possess a structure that is similar or identical to that of a peptide of the invention. As used herein, an amino acid sequence of a peptide is “similar” to that of a peptide of the invention if it satisfies at least one of the following criteria: (a) the peptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of a peptide of the present invention; (b) the peptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of a peptide sequence of the invention; or (c) the peptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding a peptide of the invention.

As used herein, a peptide with “similar structure” to that of a peptide of the invention refers to a peptide that has a similar secondary, tertiary or quaternary structure as that of a peptide of the invention. The structure of a peptide can determined by methods known to those skilled in the art, including but not limited to, X-ray crystallography, nuclear magnetic resonance, and crystallographic electron microscopy.

The term “fusion protein” as used herein refers to a peptide that comprises (i) an amino acid sequence of a peptide of the invention, a fragment thereof, a related peptide or a fragment thereof and (ii) an amino acid sequence of a heterologous peptide (i.e., not a peptide sequence of the present invention).

The term “homologue” as used herein refers to a peptide that comprises an amino acid sequence similar to that of a protein of the invention but does not necessarily possess a similar or identical function.

The term “orthologue” as used herein refers to a peptide that (i) comprises an amino acid sequence similar to that of a protein of the invention and (ii) possesses a similar or identical function.

The term “related peptide” as used herein refers to a homologue, an analogue, an isoform of, an orthologue, or any combination thereof of a peptide of the invention.

The term “derivative” as used herein refers to a peptide that comprises an amino acid sequence of a peptide of the invention which has been altered by the introduction of amino acid residue substitutions, deletions or additions. The derivative peptide possess a similar or identical function as peptides of the invention.

The term “fragment” as used herein refers to a peptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues) of the amino acid sequence of a peptide of the invention.

The term “isoform” as used herein refers to variants of a peptide that are encoded by the same gene, but that differ in their isoelectric point (pI) or molecular weight (MW), or both. Such isoforms can differ in their amino acid composition (e.g. as a result of alternative splicing or limited proteolysis) and in addition, or in the alternative, may arise from differential post-translational modification (e.g., glycosylation, acylation, phosphorylation). As used herein, the term “isoform” also refers to a protein that peptide exists in only a single form, i.e., it is not expressed as several variants.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA (1990) 87:2264-2268, modified as in Karlin and Altschul (1993) Proc. Natl. Acad. Sci. USA 90:5873-5877. The NBLAST and XBLAST programs of Altschul et al, J. Mol. Biol. (1990) 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilised as described in Altschul et al, Nucleic Acids Res. (1997) 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilised for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. Appl. Biosci. (1994) 10:3-5; and FASTA described in Pearson and Lipman Proc. Natl. Acad. Sci. USA (1988) 85:2444-8. Within FASTA, ktup is a control option that sets the sensitivity and speed of the search.

The skilled person is aware that various amino acids have similar properties. One or more such amino acids of a substance can often be substituted by one or more other such amino acids without eliminating a desired activity of that substance. Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). Substitutions of this nature are often referred to as “conservative” or “semi-conservative” amino acid substitutions.

Amino acid deletions or insertions may also be made relative to the amino acid sequence of a peptide sequence of the invention. Thus, for example, amino acids which do not have a substantial effect on the biological activity or immunogenicity of such peptides, or at least which do not eliminate such activity, may be deleted. Amino acid insertions relative to the sequence of peptides of the invention can also be made. This may be done to alter the properties of a peptide of the present invention (e.g. to assist in identification, purification or expression. Such amino acid changes relative to the sequence of a polypeptide of the invention from a recombinant source can be made using any suitable technique e.g. by using site-directed mutagenesis.

According to the various embodiments of this aspect of the invention, the promoter will preferably be of mammalian origin, but also may be from a non-mammalian animal, plant, yeast or bacteria. The promoter may be selected from but is not limited to promoter elements of the following inducible genes:
whose expression is modified in response to disturbances in the homeostatic state of DNA in the cell. These disturbances may include chemical alteration of nucleic acids or precursor nucleotides, inhibition of DNA synthesis and inhibition of DNA replication. The sequence can be selected from but not limited to the group consisting of c-myc (Hoffman et al Oncogene 21 3414-3421), p21/WAF-1 (El-Diery Curr. Top. Microbiol. Immunol. 227 121-137 (1998); El-Diery Cell Death Differ. 8 1066-1075 (2001); Dotto Biochim. Biophys. Acta 1471 43-56 (2000)), MDM2 (Alarcon-Vargas & Ronai Carcinogenesis 23 541-547 (2002); Deb & Front Bioscience 7 235-243 (2002)), Gadd45 (Sheikh et al Biochem. Pharmacol. 59 43-45 (2000)), FasL (Wajant Science 296 1635-1636 (2002)), GAHSP40 (Hamajima et al J. Cell. Biol. 84 401-407 (2002)), TRAIL-R2/DR5 (Wu et al Adv. Exp. Med. Biol. 465 143-151 (2000); El-Diery Cell Death Differ. 8 1066-1075 (2001)), BTG2/PC3 (Tirone et al J. Cell. Physiol. 187 155-165 (2001));
whose transcription is modified in response to oxidative stress. The sequence can be selected from but not limited to the group consisting of MnSOD and/or CuZnSOD (Halliwell Free Radic. Res. 31 261-272 (1999); Gutteridge & Halliwell Ann. NY Acad. Sci. 899 136-147 (2000)), IκB (Ghosh & Karin Cell 109 Suppl., S81-96 (2002)), ATF4 (Hai & Hartman Gene 273 1-11 (2001)), xanthine oxidase (Pristos Chem. Biol. Interact. 129 195-208 (2000)), COX2 (Hinz & Brune J. Pharmacol. Exp. Ther. 300 376-375 (2002)), iNOS (Alderton et al Biochem. J. 357 593-615 (2001)), Ets-2 (Bartel et al Oncogene 19 6443-6454 (2000)), FasL/CD95L (Wajant Science 296 1635-1636 (2002)), γGCS (Lu Curr. Top. Cell. Regul. 36 95-116 (2000); Soltaninassab et al J. Cell. Physiol. 182 163-170 (2000)), ORP150 (Ozawa et al Cancer Res. 61 4206-4213-(2001); Ozawa et al J. Biol. Chem. 274 6397-6404 (1999)).
whose expression is modified in response to hepatotoxic stress. The sequence can be selected from but not limited to the group consisting of Lrg-21 (Drysdale et al Mol. Immunol. 33 989-998 (1996)), SOCS-2 and/or SOCS-3 (Toilet-Egnell et al Endocrinol. 140 3693-3704 (1999), PAI-1 (Fink et al Cell. Physiol. Biochem. 11 105-114(2001)), GBP28/adiponectin (Yoda-Murakami et al Biochem. Biophys. Res. Commun. 285 372-377 (2001)), α-1 acid glycoprotein (Komori et al Biochem Pharmacol. 62 1391-1397 (2001)), metallothioneine I (Palmiter et al Mol. Cell. Biol. 13 5266-5275 (1993)), metallothioneine II (Schlager & Hart App. Toxicol. 20 395-405 (2000)), ATF3 (Hai & Hartman Gene 273 1-11 (2001)), IGFbp-3 (Popovici et al J. Clin. Endocrinol. Metab. 86 2653-2639 (2001)), VDGF (Ido et al Cancer Res. 61 3016-3021 (2001)) and HIF1α (Tacchini et al Biochem. Pharmacol. 63 139-148 (2002)).
whose expression is modified in response to a pro-apoptotic stimulus. The sequence can be selected from but not limited to the group consisting of Gadd 34 (Hollander et al J. Biol. Chem. 272 13731-13737 (1997)), GAHSP40 (Hamajima et al J. Cell. Biol. 84 401-407 (2002)), TRAIL-R2/DR5 (Wu et al Adv. Exp. Med. Biol. 465 143-151 (2000); El-Diery Cell Death Differ. 8 1066-1075 (2001)), c-fos (Teng Int. Rev. Cytol. 197 137-202 (2000)), CHOP/Gadd153 (Talukder et al Oncogene 21 4280-4300 (2002)), APAF-1 (Cecconi & Gruss Cell. Mol. Life Sci. 5 1688-1698 (2001)), Gadd45 (Sheikh et al Biochem. Pharmacol. 59 43-45 (2000), BTG2/PC3 (Tirone J. Cell. Physiol. 187 155-165 (2001)), Peg3/Pw1 (Relaix et al Proc. Nat'l Acad. Sci. USA 97 2105-2110 (2000)), Siah 1a (Maeda et al FEBS Lett. 512 223-226 (2002)), S29 ribosomal protein (Khanna et al Biochem. Biophys. Res. Commun. 277 476-486 (2000)), FasL/CD95L (Wajant Science 296 1635-1636 (2002)), tissue tranglutaminase (Chen & Mehta Int. J. Cell. Biol. 31 817-836 (1999)), GRP78 (Rao et al FEBS Lett. 514 122-128 (2002)), Nur77/NGFI-B (Winoto Int. Arch. Allergy Immunol. 105 344-346 (1994)), CyclophilinD (Andreeva et al Int. J. Exp. Pathol. 80 305-315 (1999)), p73 (Yang et al Trends Genet. 18 90-95 (2002)) and Bak (Lutz Biochem. Soc. Trans. 28 51-56 (2000)).
whose expression is modified in response to the administration of chemicals or drugs. The sequence can be selected from but not limited to the list comprised of xenobiotic metabolising cytochrome p450 enzymes from the 2A, 2B, 2C, 2D, 2E, 2S, 3A, 4A and 4B gene families (Smith et al Xenobiotica 28 1129-1165 (1998); Honkaski & Negishi J. Biochem. Mol. Toxicol. 12 3-9 (1998); Raucy et al J. Pharmacol. Exp. Ther. 302 475-482 (2002); Quattrochi & Guzelian Drug Metab. Dispos. 29 615-622 (2001)).

The promoter element may also be a synthetic promoter sequence comprised of a minimal eukaryote consensus promoter operatively linked to one or more sequence elements known to confer transcriptional inducibility in response to specific stimulus. A minimal eukaryotic consensus promoter is one that will direct transcription by eukaryotic polymerases only if associated with functional promoter elements or transcription factor binding sites. An example of which is the PhCMV*−1 (Furth et al Proc. Nat'l Acad. Sci. USA 91 9302-9306 (1994)). Sequence elements known to confer transcriptional induction in response to specific stimulus include promoter elements (Montoliu et al Proc. Nat'l Acad. Sci. USA 92 4244-4248 (1995)) or transcription factor binding sites; these will be chosen from but are not limited to the list comprising the aryl hydrocarbon (Ah)/Ah nuclear translocator (ARNT) receptor response element, the antioxidant response element (ARE), the xenobiotic response element (XRE).

A nucleic acid construct according to the invention may suitably be inserted into a vector which is an expression vector that contains nucleic acid sequences as defined above. The term “vector” or “expression vector” generally refers to any nucleic acid vector which may be RNA, DNA or cDNA.

The term “expression vector” may include, among others, chromosomal, episomal, and virus-derived vectors, for example, vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. Generally, any vector suitable to maintain, propagate or express nucleic acid to express a polypeptide in a host may be used for expression in this regard.

Recombinant expression vectors will include, for example, origins of replication, a promoter preferably derived from a highly expressed gene to direct transcription of a structural sequence as defined above, and a selectable marker to permit isolation of vector containing cells after exposure to the vector.

Expression vectors may comprise an origin of replication, a suitable promoter as defined above and/or enhancer, and also any necessary ribosome binding sites, polyadenylation regions, splice donor and acceptor sites, transcriptional termination sequences, and 5′-flanking non-transcribed sequences that are necessary for expression. Preferred expression vectors according to the present invention may be devoid of enhancer elements.

The expression vectors may also include selectable markers, such as antibiotic resistance, which enable the vectors to be propagated.

According to a second aspect of the invention there is provided a nucleic acid construct comprising a stress inducible promoter operatively isolated from a nucleic acid sequence encoding a member of the lipocalin protein family by a nucleotide sequence flanked by nucleic acid sequences recognised by a site specific recombinase, or by insertion such that it is inverted with respect to the transcription unit encoding a member of the lipocalin protein family. The recombinase recognition sites are arranged in such a way that the isolator sequence is deleted or the inverted promoter's orientation is reversed in the presence of the recombinase. The construct also comprises a nucleic acid sequence comprising a tissue specific promoter operatively linked to a gene encoding the coding sequence for the site specific recombinase.

Stress inducible promoters may be as described in relation to the first aspect of the invention.

This aspect allows for detecting reporter transgene induction in specified tissues only. By controlling the appropriate recombinase expression using a tissue specific promoter, the inducible transgene will only be viable in those tissues in which the promoter is active. For example, by driving recombinase activity from a liver specific promoter, only the liver will contain re-arranged reporter construct, and hence will the only tissue in which reporter induction can occur.

Tissue specific promoters are a class of gene promoters whose function is restricted solely (or more usually, maily) to a particular cell type or tissue.

Examples include promoters from the liver, pancreas, mammary gland, squamous epithelium, small intestine, skeletal muscle, smooth muscle, striated muscle, heart, prostate, adipose tissue, neural crest, brain, kidney and lung. Particular instances of tissue specific promoters are as follows (although, the invention is not limited as such):

| Tissue | Example of tissue specific promoter |
|---|---|
| Liver | Albumin (Pinkert et al Genes Dev 1987 1: 268-276) |
| Liver | α-fetoprotein (Wen et al DNA Cell Biol 1991 7: 525-536) |
| Liver | α1-antitrypsin (Shen et al DNA 1989 8 (2): 101-8) |
| Pancreas | Insulin II ((a) Gannon et al Genesis 2000 26(2): 139-42); |
| | (b) Ray et al Int J Pancreatol 1999 25 (3): 157-63) |
| Pancreas | Pdx-1 (Gerrish et al J Biol Chem 2000 275 (5): 3485-92) |
| Mammary gland | β-Lactoglobulin ((a) Selbert et al Transgenic Res 1998 |
| | 7 (5): 387-96); (b) Webster et al Cell Mol Biol Res 1995 |
| | 41 (1): 11-5) |
| Mammary gland | Whey acid protein (Wagner et al Nucleic Acids Res |
| | 1997 25 (21): 4323-30) |
| Squamous epithelium | Keratin 5 (Brown et al Curr Biol 1998 8 (9): 516-24) |
| Squamous epithelium | Keratin 14 (Vassar et al Proc Natl Acad Sci USA. |
| | 1989 86 (5): 1563-7) |
| Squamous epithelium | Loricrin (DiSepio et al Differentiation. 1999 64 |
| | (4): 225-35) |
| Small intestine | Fatty acid binding protein (Sweetser et al Proc Natl |
| | Acad Sci USA. 1988 85 (24): 9611-5) |
| Small intestine | sucrase-isomaltase (Markowitz et al Am J Physiol 1995 |
| | 269 (6 Pt 1): G925-39) |
| Skeletal muscle | Myosin light chain 1f (Bothe et al Genesis 2000 26 |
| | (2): 165-6) |
| Smooth muscle | SmMHC (Xin et al Physiol Genomics 2002 10 (3): 211-5) |
| Striated muscle | α-skeletal actin (Miniou et al Nucleic Acids Res 1999 |
| | 27 (19): e27) |
| Heart | α-myosin heavy chain (Heger Circ Res. 2002 90 |
| | (1): 93-9) |
| Prostate | Probasin (Greenberg et al Mol Endocrinol 1994 8: 230-239) |
| Adipose tissue | aP2 (Gnudi et al Am J Physiol 1996 270 (4 Pt 2): R785-92) |
| Neural crest | Pax3 (Goulding et al EMBO J 1991 10 (5): 1135-47) |
| Neural crest | Protein 0 (Yamauchi et al Dev Biol 1999 212 (1): 191-203) |
| Brain | CaMKII (Tomioka et al Brain Res Mol Brain Res 2002 |
| | 108 (1-2): 18-32) |
| Lung | surfactant protein C (Korfhagen et al J Clin Invest 1994 93 (4): |
| | 1691-9) |

The recombination event producing an active reporter transcription unit may therefore only take place in tissues where the recombinase is expressed. In this way the reporter may only be expressed in specified tissue types where expression of the recombinase results in a functional transcription unit comprised of the inducible promoter linked to the promoter. Site specific recombinase systems know to perform such a function include the bacteriophage P1 cre-lox and the bacterial FLIP systems. The site specific recombinase sequences may therefore be two loxP sites of bacteriophage P1

The use of site specific recombination systems to generate precisely defined deletions in cultured mammalian cells has been demonstrated. Gu et al. (Cell 73 1155-1164 (1993)) describe how a deletion in the immunoglobulin switch region in mouse ES cells was generated between two copies of the bacteriophage P1 loxP site by transient expression of the Cre site-specific recombinase, leaving a single loxP site. Similarly, yeast FLP recombinase has been used to precisely delete a selectable marker defined by recombinase target sites in mouse erythroleukemia cells (Fiering et al., Proc. Nat'l. Acad. Sci. USA 90 8469-8473 (1993)). The Cre lox system is exemplified below, but other site-specific recombinase systems could be used.

A construct used in the Cre lox system will usually have the following three functional elements:
1. The expression cassette;
2. A negative selectable marker (e.g. Herpes simplex virus thymidine kinase (TK) gene) expressed under the control of a ubiquitously expressed promoter (e.g. phosphoglycerate kinase (Soriano et al., Cell 64 693-702 (1991)); and
3. Two copies of the bacteriophage P1 site specific recombination site loxP (Baubonis et al. Nuc. Acids. Res. 21 2025-2029 (1993)) located at either end of the DNA fragment.

This construct can be eliminated from host cells or cell lines containing it by means of site specific recombination between the two loxP sites mediated by Cre recombinase protein which can be introduced into the cells by lipofection (Baubonis et al., Nuc. Acids Res. 21 2025-2029 (1993)). Cells which have deleted DNA between the two loxP sites are selected for loss of the TK gene (or other negative selectable marker) by growth in medium containing the appropriate drug (ganciclovir in the case of TK).

According to the third aspect of the invention there is provided a host cell transfected with a nucleic acid construct according to any one of the previous aspects of the invention. The cell type is preferably of human or non-human mammalian origin but may also be of other animal, plant, yeast or bacterial origin. For example, HEPA1-6, mouse hepatoma epithelial cells; 1293, human embryonic kidney epithelial cells; COS-1, African green monkey fibroblasts; CHO, Chinese hamster ovary epithelial cells; HT 29, human colon adenocarcinoma epithelial cells; MCF7, human breast adenocarcinoma epithelial-like cells; HeLa, human cervical carcinoma epithelial cells, HEP G2, human hepatocyte carcinoma epithelial cells; PC3, human prostate adenocarcinoma epithelial cells; A2780, human ovarian carcinoma epithelial cells.

Introduction of an expression vector into the host cell can be effected by calcium phosphate transfection, DEAE-dextran mediated transfection, microinjection, cationic lipid-mediated transfection, electroporation, transduction, scrape loading, ballistic introduction, infection of other methods. Such methods are described in many standard laboratory manuals, such as Sambrook et al., Molecular Cloning: A Laboratory Manual, 2^{nd} Ed., Cold Spring Harbor Laboratory Press, Cold Spring Harbor, N.Y. (1989).

According to the fourth aspect of the invention, there is provided a transgenic non-human animal in which the cells of the non-human animal express the protein encoded by the nucleic acid construct according to any one of the previous aspects of the invention. Suitably, the non-human animal is a non-human mammal. The transgenic animal is preferably a mouse but may be another mammalian species, for example another rodent, e.g. a rat or a guinea pig, or another species such as rabbit, or a canine or feline, or an ungulate species such as ovine, porcine, equine, caprine, bovine, or a non-mammalian animal species, e.g. an avian (such as poultry, e.g. chicken or turkey).

In embodiments of the invention relating to the preparation of a transfected host cell or a transgenic non-human animal comprising the use of a nucleic acid construct as previously described, the cell or non-human animal may be subjected to further transgenesis, in which the transgenesis is the introduction of an additional gene or genes or protein-encoding nucleic acid sequence or sequences. The transgenesis may be transient or stable transfection of a cell or a cell line, an episomal expression system in a cell or a cell line, or preparation of a transgenic non-human animal by pronuclear microinjection, through recombination events in embryonic stem (ES) cells or by transfection of a cell whose nucleus is to be used as a donor nucleus in a nuclear transfer cloning procedure.

Methods of preparing a transgenic cell or cell line, or a transgenic non human animal, in which the method comprises transient or stable transfection of a cell or a cell line, expression of an episomal expression system in a cell or cell line, or pronuclear microinjection, recombination events in ES cells, or other cell line or by transfection of a cell line which may be differentiated down different developmental pathways and whose nucleus is to be used as the donor for nuclear transfer; wherein expression of an additional nucleic acid sequence or construct is used to screen for transfection or transgenesis in accordance with the first, second, third, or fourth aspects of the invention. Examples include use of selectable markers conferring resistance to antibiotics added to the growth medium of cells, e.g. neomycin resistance marker conferring resistance to G418. Further examples involve detection using nucleic acid sequences that are of complementary sequence and which will hybridise with, or a component of, the nucleic acid sequence in accordance with the first, second, third, or fourth aspects of the invention. Examples would include Southern blot analysis, northern blot analysis and PCR.

According to the fifth aspect of the invention, there is provided the use of a nucleic acid construct in accordance with any one of the first, second, third, or fourth aspects of the invention for the detection of a gene activation event resulting from a change in altered metabolic status in a cell in vitro or in vivo.

The gene activation event may be the result of induction of toxicological stress, metabolic changes, or disease that may be, but is not limited to, the result of viral, bacterial, fungal or parasitic infection.

According to the sixth aspect of the invention there is provided the use of a nucleic acid construct comprising a nucleic acid sequence encoding a member of the lipocalin protein family, wherein said lipocalin protein is heterologous to the cell in which it is expressed, for the detection of a gene activation event resulting from a change in altered metabolic status in a cell in vitro or in vivo.

The gene activation event may be the result of induction of toxicological stress, metabolic changes, disease that may be, but is not limited to, the result of viral, bacterial, fungal or parasitic infection.

Uses in accordance with the fifth and sixth aspects of the invention also extend to the detection of disease states or characterisation of disease models in a cell, cell line or non human transgenic animal where a change in the gene expression profile within a target cell or tissue type is altered as a consequence of the disease. Diseases in the context of this aspect of the invention which are detectable under the methods disclosed may be defined as infectious disease, cancer, inflammatory disease, cardiovascular disease, metabolic disease, neurological disease and disease with a genetic basis.

An additional use in accordance with this aspect of the invention involves the growth of a transfected cell line in accordance with the third aspect in a suitable immunocompromised mouse strain (referred to as a xenograft), for example, the nude mouse, wherein an alteration in the expression of the reporter described in the first or second aspects of the invention may be used as a measure of altered metabolic status of the host as a result of toxicological stress, metabolic changes, disease with a genetic basis or disease that may be, but is not limited to, the result of viral, bacterial, fungal or parasitic infection. The scope of this use may also be of use in monitoring the effects of exogenous chemicals or drugs on the expression of the reporter construct.

The fifth and sixth aspects of the invention extend to methods of detecting a gene activation event in vitro or in vivo.

In an embodiment according to the fifth aspect of the invention, the method comprises assaying a host cell stably transfected with a nucleic acid construct in accordance with any one of the first or second aspects of the invention, or a transgenic non-human animal according to the fourth aspect of the invention, in which the cell or animal is subjected to a gene activation event that is signalled by expression of a peptide tagged lipocalin reporter gene.

In an embodiment according to the sixth aspect of the invention, the method comprises assaying a host cell stably transfected with a nucleic acid construct comprising a nucleic acid sequence encoding a member of the lipocalin protein family, wherein said lipocalin protein is heterologous to the cell in which it is expressed, or a transgenic non-human animal whose cells express such a construct, in which the cell or animal is subjected to a gene activation event that is signalled by expression of a peptide tagged lipocalin reporter gene.

Accordingly there is provided a method of screening for, or monitoring of toxicologically induced stress in a cell or a cell line or a non-human animal, comprising the use of a cell, cell line or non human animal which has been transfected with or carries a nucleic acid construct as described above.

Toxicological stress may be defined as DNA damage, oxidative stress, post translational chemical modification of cellular proteins, chemical modification of cellular nucleic acids, apoptosis, cell cycle arrest, hyperplasia, immunological changes, effects consequent to changes in hormone levels or chemical modification of hormones, or other factors which could lead to cell damage.

Accordingly, there is also provided a method for screening and characterising viral, bacterial, fungal, and parasitic infection comprising the use of a cell, cell line or non human animal which has been transfected with or carries a nucleic acid construct as described above.

Accordingly, there is additionally provided a method for screening for cancer, inflammatory disease, cardiovascular disease, metabolic disease, neurological disease and disease with a genetic basis comprising the use of a cell, cell line or non human animal which has been transfected with or carries a nucleic acid construct as described above.

In these contexts the cell may be transiently transfected, maintaining the nucleic acid construct as described above episomally and temporarily. Alternatively cells are stably transfected whereby the nucleic acid construct is permanently and stably integrated into the transfected cells' chromosomal DNA.

Also in this context transgenic animal is defined as a non human transgenic animal with the nucleic acid construct as defined above preferably integrated into its genomic DNA in all or some of its cells.

Expression of the peptide tagged lipocalin protein in respect of the fifth aspect of the invention can be assayed for by measuring levels of the lipocalin protein in cell culture medium or purified or partially purified fractions thereof.

Lipocalins are known to be secreted into body fluids and some are known to be eliminated in urine. Expression of the peptide tagged lipocalin protein in accordance with the fourth aspect of the invention therefore can be assayed for by measuring levels of lipocalin secreted into harvestable body fluids. In a preferred embodiment of the invention the body fluid will be urine, but may also be selected from the list including milk, saliva, tears, semen, blood and cerebrospinal fluid, or purified or partially purified fractions thereof.

Detection and quantification of the tagged lipocalins secreted from cultured cells into tissue culture medium or transgenic non-human animal body fluid may be achieved using a number of methods known to those skilled in the art:

1. Immunological Methods.

(i) The assay may be an ELISA whereby an antibody or antiserum containing a single or mixture of antibodies recognising either the lipocalin reporter itself or the peptide tag attached to and is used as a capture antibody to coat a microtitre plate or other medium suitable for conducting the assay. The culture medium or body fluid containing the reporter gene product (analyte) is added to the microtitre plate to allow binding of the analyte. Addition of the same antibody or antiserum that has been conjugated to an enzyme, commonly horseradish peroxidase, is used as a second antibody. Addition of a suitable substrate, preferably one producing a colour product following conversion by the enzyme is used to quantify the analyte in proportion to how much second antibody conjugate has been bound.

(ii) Competitive ELISA. In an alternative form the tissue culture medium or the body fluid (analyte) sample containing the tagged lipocalin is bound to a support suitable for conducting the assay. In a separate reaction a limited standard amount of antibody specifically recognising the reporter gene product is added to a separate aliquot of the same and allowed to bind. This is added to the analyte bound the support to allow remaining free antibody to bind. A second, enzyme conjugated antibody against for example the Fc region of the first antibody is allowed to bind and the calorimetric readout can be used to quantify the analyte whereby the degree of colour change is inversely proportional to the level of analyte in the sample.

(iii) Western Blot Analysis

Transfected cell homogenates were prepared by incubation of cells in homogenization buffer (140 mM NaCl, 50 mM Tris-HCl pH7-5, 1 nM EDTA, 1% Triton-100) for 30 minutes on ice. Following a brief centrifugation to remove insoluble material the cleared supernatants were assayed for protein content. A volume equivalent to 40 μg cell extract and an equal volume of cell medium were subjected to SDS-PAGE and blotted onto nitrocellulose (Schleicher and Schuell, Dassel, Germany) membrane using a semi-dry blotting apparatus (Bio-Rad, Richmond, Calif.). The membranes were blocked for 1 hour in blocking buffer (5% NFDM w/v in PBS) then incubated with myc mAb Invitrogen Life Technologies, Carlsbad, Calif.) diluted in blocking buffer for 2 hours with continuos agitation. After a series of washes in PBST (PBS plus 0.05% Tween-20), the membrane was incubated in an anti-mouse antibody conjugated to HRP diluted in blocking buffer for one hour with agitation, and after another series of washes in PBST the HRP activity was developed using an ECL kit (Pierce, Rockford, Ill.) and captured on autoradiographic film (Kodak).

(iv) Fluorescence polarisation. The antibody specifically recognising the reporter lipocalin protein is conjugated with fluorescein and mixed with the analyte produced. This method quantifies the analyte by direct measurement of the amount of antibody-antigen complex present. This method may also be adapted to measure any protein-protein interaction.

2. Release of a Labelled Substrate. E.g. Radioactive (CAT) or Fluorometric, Colorimetric.

Detection of conversion of substrate due to enzymatic activity of the lipocalin reporter protein produced. The nature of substrate conversion may or may not fall into one or more of the following event categories: Proteolysis, phosphorylation, acetylation, sulphation, methylation

3. Detection of Multiple Substrates. Where a multiple of lipocalin reporter proteins are used methods suitable for detection of such events could include but not necessarily be limited to:

(i) Mass Spectrometry

(ii) Nuclear Magnetic Resonance (NMR)

In a preferred embodiment of the invention there is provided a method of detecting a reporter gene activation event, comprising the steps of:
1. Transfecting a cell or microinjecting the pronucleus of a fertilised mouse egg with a nucleic acid sequence encoding a lipocalin protein tagged with a peptide or protein as described above in accordance with the first, second, third, or fourth aspects of the invention. Optionally use the microinjected egg or transfected mouse ES cell line;
2. Exposing the transfected cell, cell line or transgenic non human animal to a stimulus which may or may not cause a change in metabolic status resulting alteration in gene expression; and
3. Using a suitable assay to determine the level expression of the tagged lipocalin reporter, for example using detection methods such as ELISA, RIA, Mass spectrometry, NMR, telemetric methods.

In step (1), the detectable lipocalin protein may be a heterologous protein to the cell in which the nucleic acid construct is expressed. Such an “untagged” lipocalin reporter protein may not therefore need a peptide or protein tag for detection.

Methods and uses in accordance with the present invention offer significant advances in investigating any area in which modified gene expression plays a significant role. Such peptide tagged lipocalin genes will be of use in cells and transgenic animals to detect activity of selected genes. Specific applications include but are not restricted to:
1. Providing a rapid and robust in vivo screening system for assessing the potential toxic effects of chemicals.
2. Provide information on the mechanism of toxicity. Such information could be used to eliminate compounds from a selection process or suggest possible modifications to a compound.
3. Provide information on the effect of combinations of compounds.
4. Allow monitoring of variation in reporter gene expression over time by measuring levels of reporter(s) in urine at different time intervals.
5. Assessment of changes in gene expression associated with pathogenic infection.
6. Assessment of changes in gene expression associated with neurological, cardiovascular and metabolic diseases.
7. Assessment of changes in gene expression associated with cancer.
8. Provide information allowing validation of drug target selection e.g. by matching reporter expression profile to actions of toxins whose mechanism is defined and understood.
9. Use for evaluating compounds as therapeutic strategies aimed at reversing a toxic, metabolic, or degenerative phenotype.
10. Assessment of changes in gene expression resulting from environmental and/or behavioural changes.

Preferred features for the second and subsequent aspects of the invention are as for the first aspect mutatis mutandis.

The present invention will now be described with reference to the following examples which are present for the purposes of illustration only and should no be construed as being limited with respect to the invention. Reference in the application is also made to a number of drawings in which:

FIG. 1 shows the position of the peptide tag at the amino terminal or carboxy terminal or inserted internally with respect to the amino acid sequence of the lipocalin reporter protein

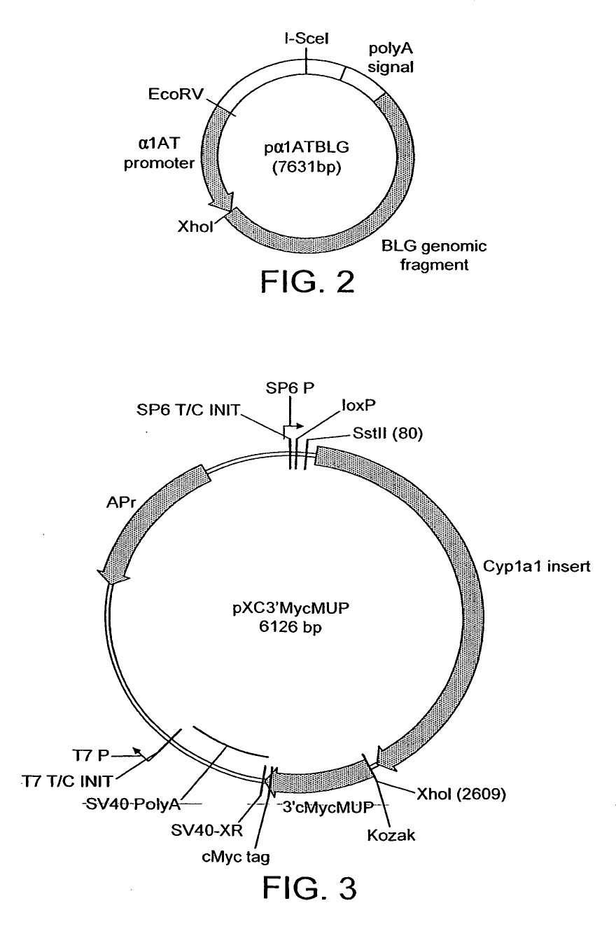

FIG. 2 shows the plasmid map for pα1ATBLG

FIG. 3 shows the plasmid map for pXC3′MycMUP

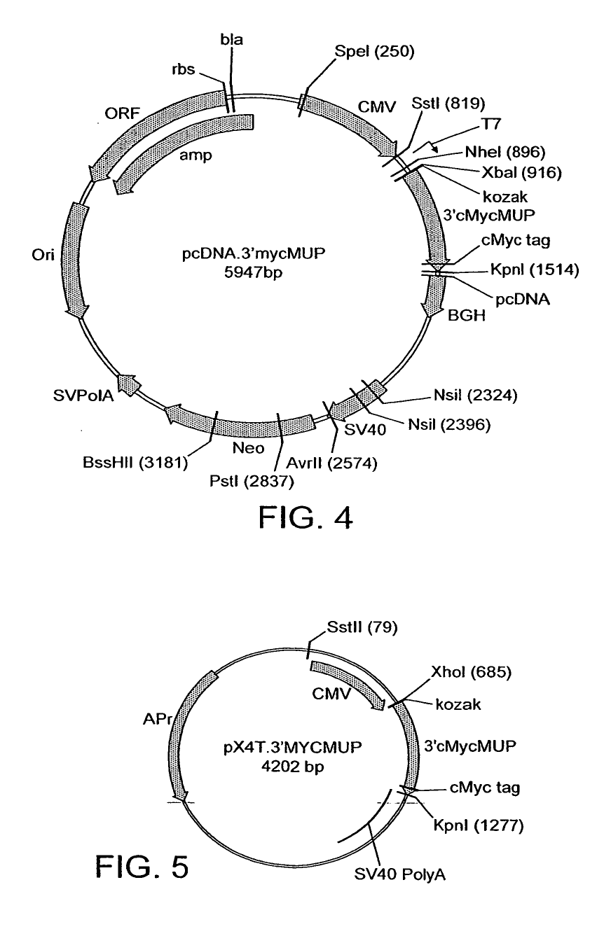

FIG. 4 shows the plasmid map for pcDNA.3′mycMUP

FIG. 5 shows the plasmid map for pX4T.3′MYCMUP

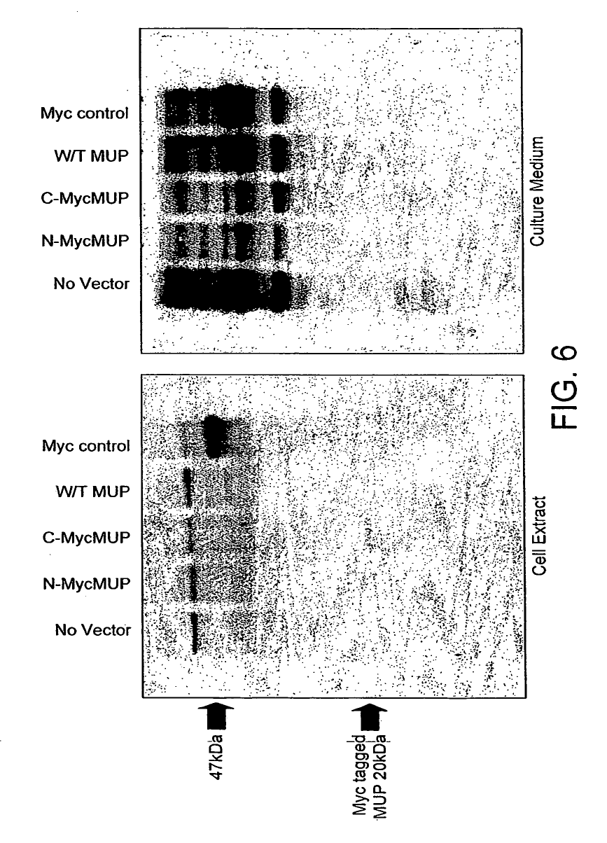

FIG. 6 shows the results of expression of Myc tagged MUP

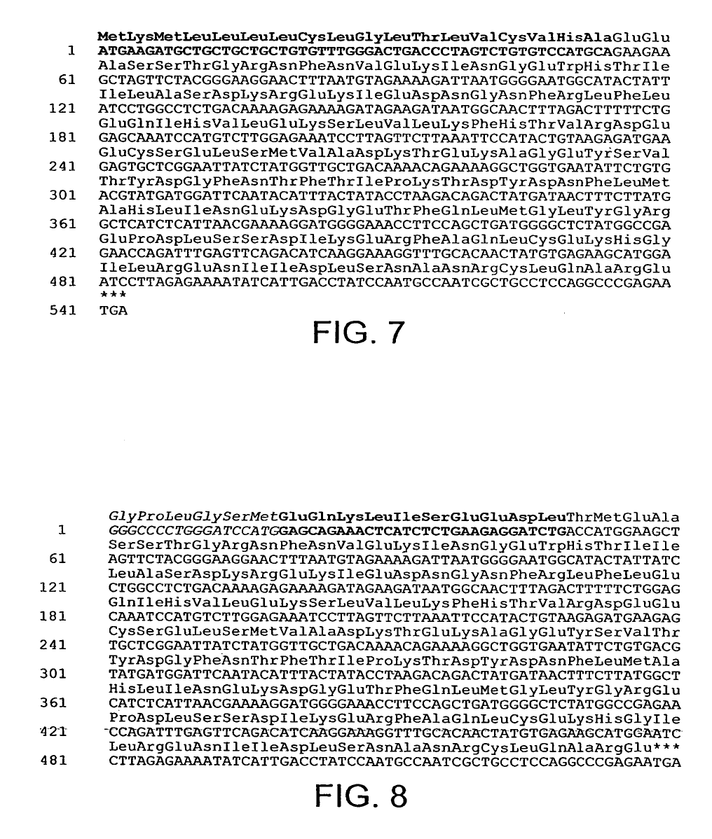

FIG. 7 shows the DNA and amino acid sequences of the MUP clone Mmup9a. The 18 amino acid secretion signal peptide is shown in bold (amino acid residues 1 to 18).

FIG. 8 shows the DNA and amino acid sequence of the recombinant mMUP reporter molecule. The protein contains a sixteen amino acid N-terminal addition, comprising of 6 amino acids from the pGEX vector (italics—amino acid residues 1 to 6) and the c-myc epitope (shown in bold—amino acid residues 7 to 16).

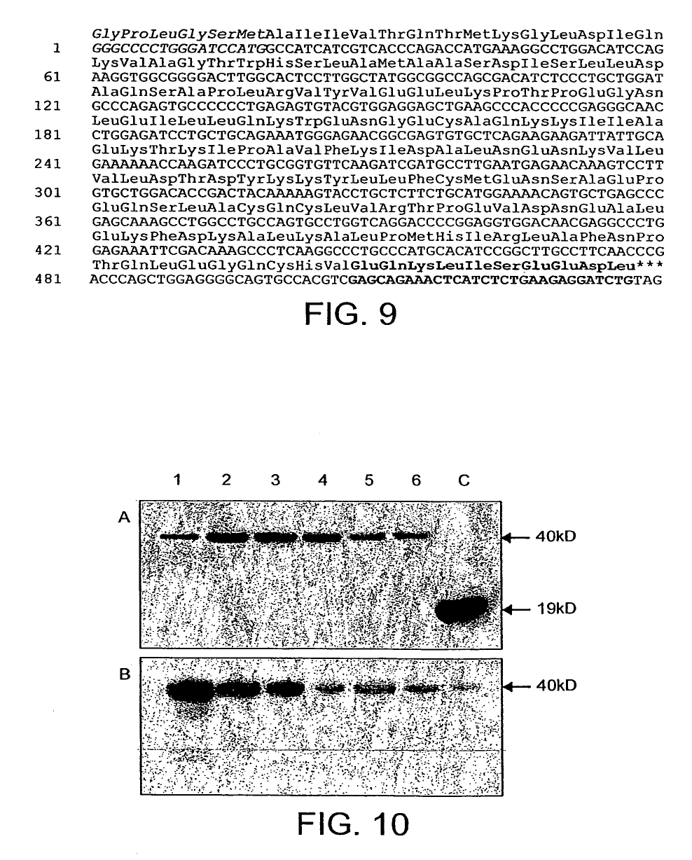

FIG. 9 shows the DNA and amino acid sequence of the recombinant BLGm reporter molecule. The protein contains a six amino acid N-terminal addition from the pGEX vector (italics—amino acid residues 1 to 6) and the C-terminal c-myc epitope (bold—amino acid residues 170 to 179).

FIG. 10 shows (a) Western blot of GST-BLGm fusion protein. Lanes 1 to 6 show fractions eluted from a glutathione-agarose column. Lane C, MUP protein control. (b) Western blot of GST-MUPm fusion protein. Lanes 1 to 7 show fractions eluted from glutathione-agarose column. Blots were probed using 9E10 anti-myc antibody directly conjugated to URP (Roche).

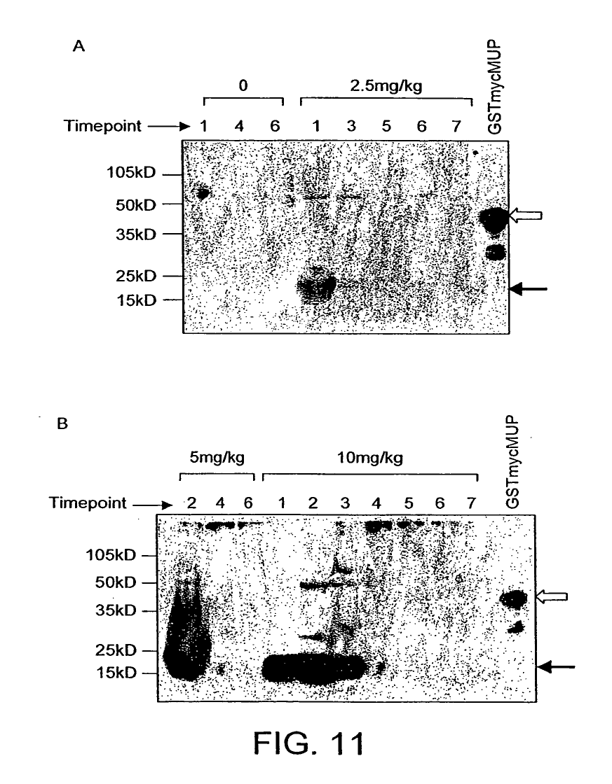

FIG. 11 shows Western blot analysis of urine samples (15 μl) collected from mice, following injection with either (A) vehicle or recombinant mMUP (2.5 mg/kg); or (B) recombinant mMUP (5 and 10 mg/kg). Blots were probed with anti-myc antibody. Uninjected recombinant GSTmMUP (˜45 kDa, open arrow) was included as a positive control (right hand lane). The closed arrow indicates the position of the ˜18 kDa mMUP control band.

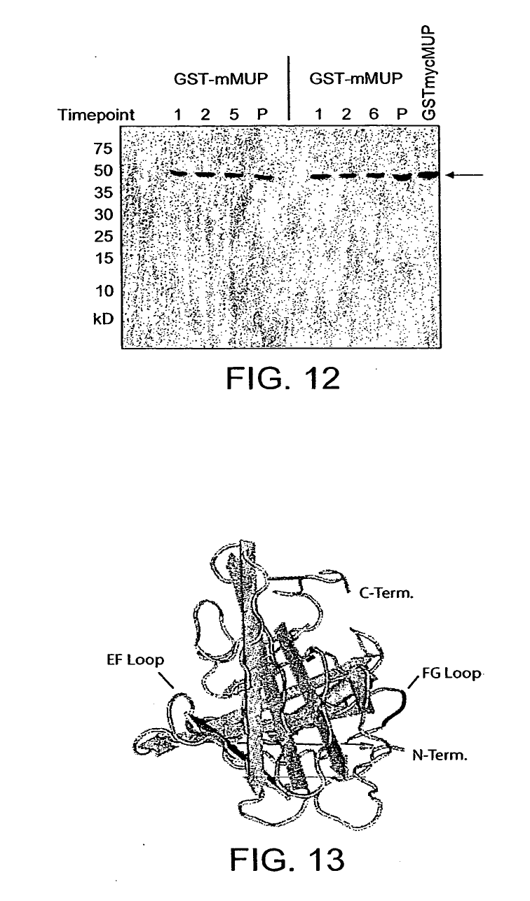

FIG. 12 shows Western blot analysis of urine samples taken at various time points (in hours) and plasma (P) at 24 hours from mice that had been injected with recombinant GST-BLGm and GST-mMUP. Blots were probed with an anti-GST antibody. Arrow indicates the expected size of the band corresponding to GST-mMUP protein.

FIG. 13 shows the 3-dimensional solution structure of MUP. The antiparallel β-sheets are shown in brown, and the loop regions in blue. The BF loop is marked, as is the FG loop. Red lines indicate amino acid positions where the internal restriction site additions were made.

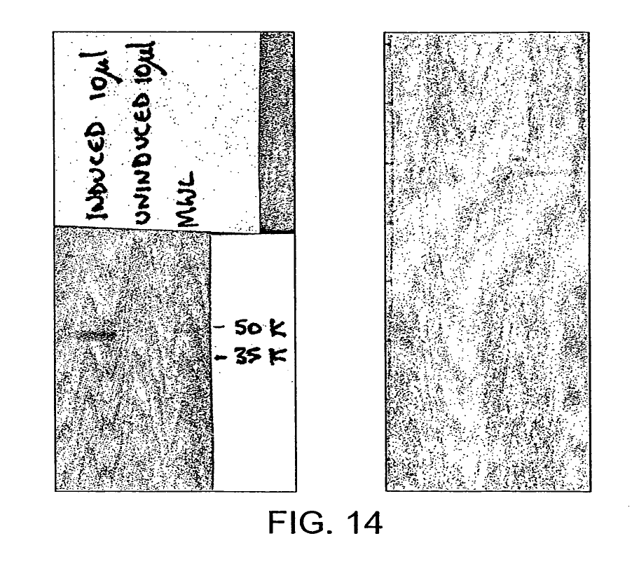

FIG. 14 shows antibody detection of epitope tagged MUP reporter proteins: (A) Haemaglutinin (HA) tagged MUP protein was expressed in E. coli, and extracts from induced (Lane 1) and uninduced (Lane 2) cells analysed by western blotting using an anti-HA antibody (3F10, Roche) HRP conjugated second antibody and ECL detection (Amersham). Lane 3 contains molecular size markers. A specific band of the expected size is seen for the HA-tagged GST-MUP fusion protein; (B) ERB tagged MUP protein was expressed in E. coli and extracts from induced (Lane 2) and uninduced (Lane 3) cells analysed by western blotting using an anti-ERB antibody (ICRF Technology), HRP-conjugated second antibody and ECL detection (Amersham). (Lane 1 molecular size markers). A specific band of the expected size is seen for the ERB-tagged GST-MUP fusion protein. Extensive photo-bleaching is seen in Lane 1, due to the amount of protein present.

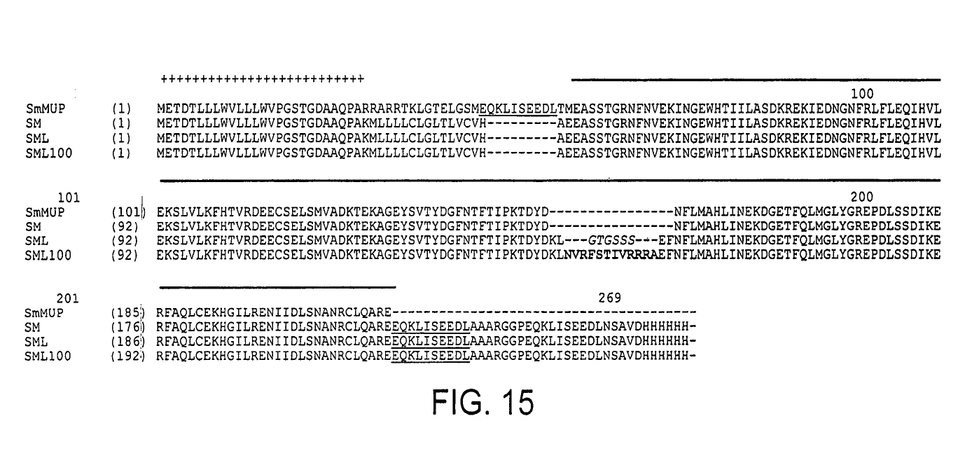

FIG. 15 shows modified MUP proteins produced from the pSecTag vector. The various modifications made to the wild-type MUP protein sequence (overlined region) are shown: the Igκ signal peptide leader, which is cleaved during processing (++++); the c-myc epitope tag (underlined); the iTag insertion sequence in the FG loop (italics); and the Clone 100 epitope tag (bold), and the other C- and N-terminal modifications and additions.

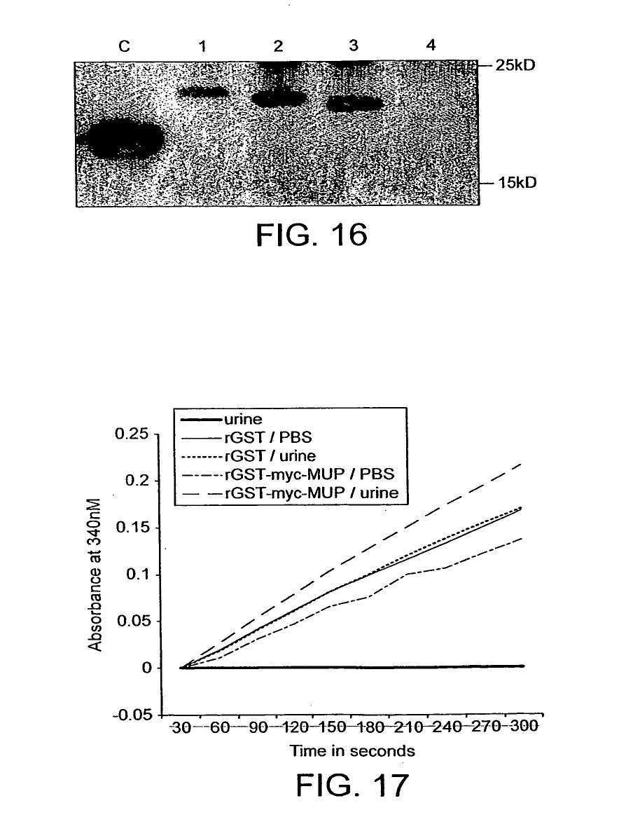

FIG. 16 shows results of pSecTag MUP constructs that were transfected into A2780 cells using Fugene, and the medium (50 μl) directly examined for secreted protein by Western blotting, using anti-myc antibody 9E10. Lane C, recombinant mMUP control; Lane 1, pSML.ic100; Lane 2, pSML; Lane 3, pSM; Lane 4, pSecmMUP. Several protein bands are present in the pSecmMUP medium, due to the presence of multiple start sites in the 5′-region of this construct.

FIG. 17 shows analysis of mouse urine containing either GST or GST-mMUP, together with GST or GST-mMUP in phosphate buffered saline (PBS) for GST enzymic activity. The concentration of all proteins was 100 μg/ml. The graph shows GST enzymic activity, as absorbance (340 nm) versus time, relative to the absorbance at the 30 second timepoint.

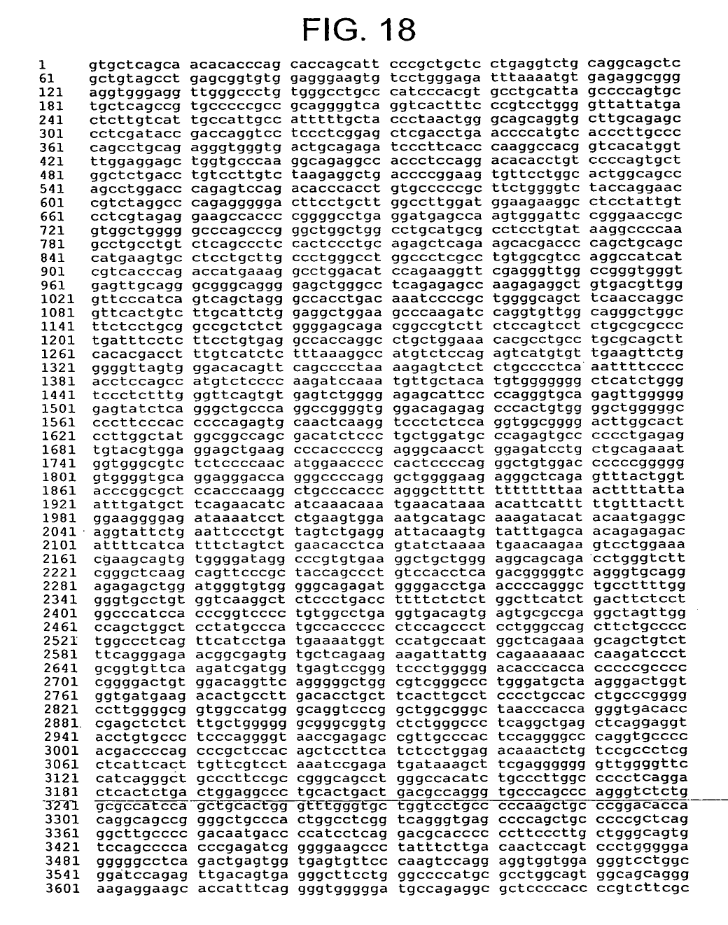

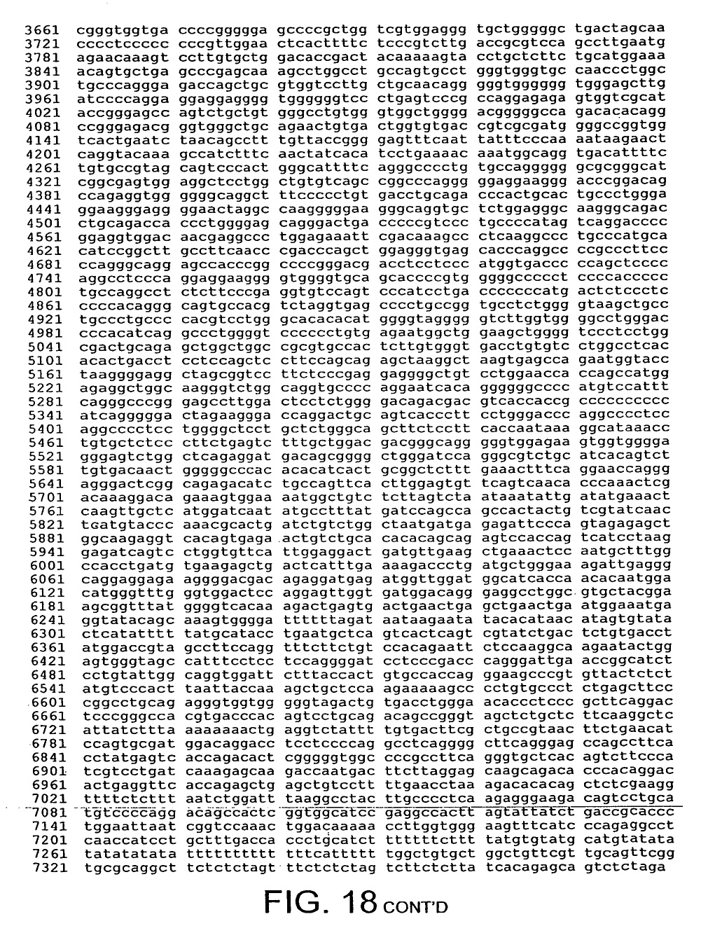

FIG. 18 shows the nucleotide sequence for ovine betalactoglobulin (BLG) (accession no. X12817), available from www.ncbi.nlm.nih.gov/entrz, published by Harris, S et al Nucleic Acids Res. 16 (21), 10379-10380 (1988); Watson, C. J. et al Nucleic Acids Res. 19 (23), 6603-6610 (1991). The signal peptide is coded for by residues 842 to 895 and mature protein from 6 exons at residues 896 . . . 937, 1602 . . . 1741, 2586 . . . 2659, 3772 . . . 3882, 4551 . . . 4655, 4869 . . . 4882

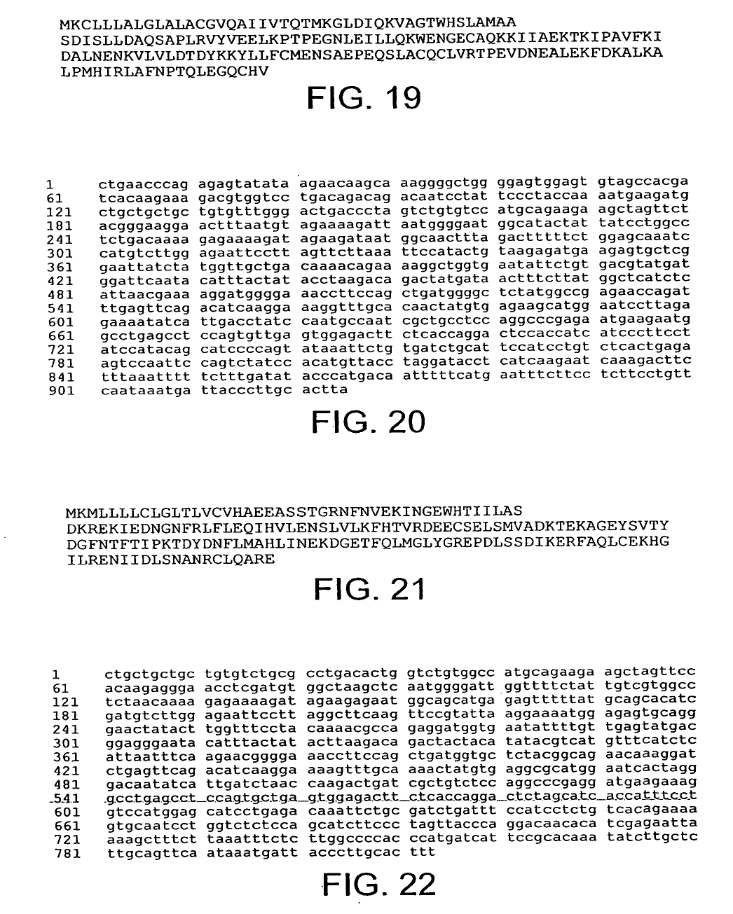

FIG. 19 shows the amino acid sequence for ovine betalactoglobulin (BLG) coded for by the nucleotide sequence of FIG. 16.

FIG. 20 shows the cDNA encoding the mRNA of murine major urinary protein 1 (Mup1), (Accession no. NM 031188), ), available from www.ncbi.nlm.nih.gov/entrz, published Lucke et al Eur. J. Biochem. 266 (3), 1210-1218 (1999); Abbate, et al J. Biomol. NMR 15 (2), 187-188 (1999); Ferrari et al FEBS Lett. 401 (1), 73-77 (1997); Held, et al Mol. Cell. Biol. 7 (10), 3705-3712 (1987); Bennett et al J. Cell Biol. 105 (3), 1073-1085 (1987); Shahan et al Mol. Cell. Biol. 7 (5), 1938-1946 (1987); Clark et al EMBO J. 4 (12), 3167-3171 (1985); Clark, et al EMBO J. 4 (12), 3159-3165 (1985); Ghazal et al Proc. Nat'l. Acad. Sci. USA. 82 (12), 4182-4185 (1985); Kuhn et al Nucleic Acids Res. 12 (15), 6073-6090 (1984); Clark et al EMBO J. 3 (5), 1045-1052 (1984); Krauter et al J. Cell Biol. 94 (2), 414-417 (1982); coding sequence from residues 112 . . . 654.

FIG. 21 shows the amino acid sequence for murine major urinary protein coded for by the nucleotide sequence of FIG. 18.

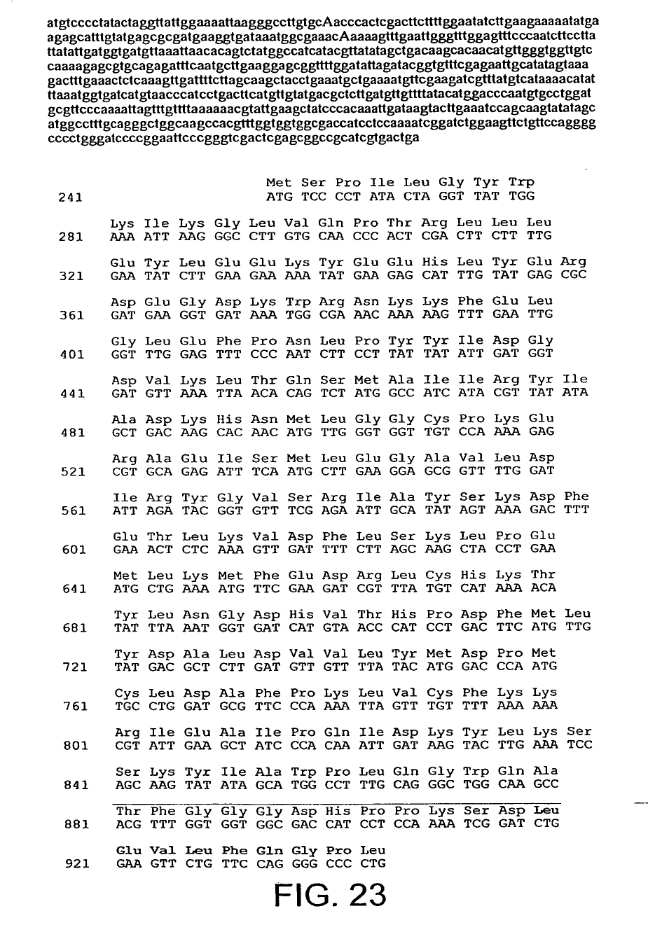

FIG. 22 shows the cDNA sequence encoding the mRNA of rat alpha-2-u globulin (accession no. M27434)), available from www.ncbi.nlm.nih.gov/entrz, published by Roy et al J. Steroid Biochem. 27 (4-6), 1129-1134 (1987)

FIG. 23 shows the GST coding sequence derived from pGEX6p-1. The GST coding sequence is nucleotide residues 241-917. The residues highlighted in bold

Leu Glu Val Leu Phe Gln Gly Pro
ctg gaa gtt ctg ttc cag ggg ccc

represent the PreScission™ Protese cleavage recognition sequence position 918-938. The protease cleavage site allows for the production of cleaved myc-tagged proteins from the GST fusion proteins as described in Example 6.

### EXAMPLE 1

### Preparation of pα1ATBLG

The α1AT promoter (350 bp) was excised from a1AT/CAT (Yull et al Transgenic Res. 4 70-74 (1995)) as a HindIII SmaI fragment and inserted into pBlueα1AT. Digestion of this with EcoRV and XhoI allowed direct insertion of the α1AT promoter into pXen6.S (Simon Temperley, CXR Biosciences) digested with the same enzymes. The microinjection fragment was purified after digestion of the plasmid with pα1ATBLG (shown in FIG. 2).

### EXAMPLE 2

### Preparation of pX4T3′MycMUP

A XhoI/KpnI fragment encoding amino terminal c-Myc tagged mouse MUP was inserted into pXAM4 (CXR Biosciences) effectively placing it under the control of the CMV promoter. pXAM4 was previously constructed by inserting a PCR generated fragment containing the CMV promoter as a BamHI-XhoI fragment into a pSP72 (Promega) multiple cloning site which had been modified by addition of a linker which added restriction sites allowing insertion of additional fragments downstream of the CMV promoter sequence.

### EXAMPLE 3

### Preparation of pXC3′MvcMUP

A 2.5 kb DNA fragment encompassing the murine Cyp1A1 promoter and upstream sequences was inserted into SstII/XhoI digested pX4T.3′MycMUP (Thomas McCartney, CXR Biosciences) to engineer a reporter vector capable of expressing COOH terminally c-Myc tagged MUP upon induction of the CYP1A1 promoter using a suitable inducing agent, if the construct is used to transfect a suitable cell line or to generate a transgenic animal.

### EXAMPLE 4

### pcDNA.3′MycMWP

A DNA fragment encompassing the COOH terminally c-Myc tagged MUP was excised from pX4T.3′Myc (Thomas McCartney, CXR Biosciences) to engineer an expression vector capable of constitutive expression of c-Myc tagged MUP if used to transfect a suitable cell line or to generate a transgenic animal.

### EXAMPLE 5

### Expression of Myc-MUP

Constructs were tested by transient transfection of a 90% confluent monolayer of Hepa1-6 cells in a T-25 flask using 6 ug of DNA in accordance with the protocol supplied with Lipofectamine transfection reagent (Invitrogen).

Cells and 5 ml of medium were harvested 48 hours post-transfection. Total protein from the cell pellets was obtained using 1 ml TR1 reagent (Sigma) per pellet in accordance with directions. Cellular protein was further purified using the PlusOne SDS-PAGE Clean-Up Kit (Amersham) in accordance with directions. Correspondingly, protein was purified from 100 μl samples of growth medium from each transfected cell batch using the PlusOne SDS-PAGE Clean-Up Kit in accordance with directions.

Cell extracts and culture medium from Hepa1 cells transfected with constructs designed to constitutively express NH3 and COOH terminally Myc tagged MUP coding sequences from the CMV promoter (2^{nd} and 3^{rd} lanes from left respectively in both left and right panels; plasmids X4T5′MycMUP and X4T3′MycMUP respectively) were subject to SDS-PAGE. Results shown in FIG. 6

Western blot analysis by probing with antibody against c-Myc showed the presence of COOH terminally tagged MUP in both cell extract and medium of Hepa1 cells (3^{rd} lane from left in both left and right hand panels). Results shown in FIG. 6

25% of the total cellular protein samples and the entire protein sample derived from the growth medium were analysed by SDS-PAGE followed by western blot in accordance with equipment manufacturer's (BIO-RAD) directions. The blot was probed using the murine monoclonal Anti-Myc antibody 9E10 (Sigma) in conjunction with anti-mouse Ig HRP conjugated antibody (Amersham). Visualisation was performed using ECL reagent (Amersham) in accordance with directions.

### EXAMPLE 6

### Production of Recombinant Epitope Tagged Lipocalin Proteins

Two candidate lipocalin family members, ovine beta-lactoglobulin (BLG) and mouse major urinary protein (MUP) have been shown to function as excreted reporter molecules. This has been achieved by introducing recombinant protein to mice via intravenous injection into the tail vein, followed by analysis of urine and plasma by western blotting.

To expand the application of a secreted/excreted reporter, it is possible to modify the reporter protein by the addition of specific epitope tag. This should allow a single reporter protein backbone to report on a number of specific events within a single system. We have demonstrated the ability to introduce additional amino acid motifs containing epitope tags at the N-terminus, the C-terminus and at several internal loop positions of the lipocalin reporter protein.

Recombinant MUP and BLG were expressed in E. coli using the pGEX vector system (Amersham Bioscience), which expresses all inserted sequences as a C-terminal fusion protein with vector encoded glutathione-S-transferase (GST). GST may be removed from the inserted fusion partner via a specific proteolytic cleavage site located at the C terminal end of GST.

A MUP clone, Mmup9a, was derived from mouse liver RNA by RT-PCR, and the identity confirmed by sequencing (FIG. 7). This clone, Mmup9a, is almost identical (536/537 bases) to the MusMup1 type I MUP clone (M16355, Genbank). The MUP coding sequence, minus the N-terminal 18 amino acid signal peptide, was rederived from clone Mup9a, by PCR as an NcoI-XhoI fragment, and cloned into the E. coli expression vector pGEX-6PB (derived from pGEX-6P-1, Amersham Bioscience) to produce pGEX-MUP. A synthetic linker oligonucleotide was then used to add the c-myc epitope sequence, as an NcoI-NcoI fragment, to the 5′-end of the MUP coding sequence to give pGEX-mMUP.

pCD3′mycBLG, containing the BLG precursor protein cDNA fused with a C-terminal myc epitope tag, was constructed from the BLG cDNA clone pBlacD (Roslin Institute). The C-terminal myc-tagged BLG coding sequence, minus the 18 amino acid signal peptide, was derived by PCR from pCD3′mycBLG (containing the BLG precursor protein cDNA fused with a C-terminal myc epitope tag) and cloned directly into pGEX-6PB, to produce pGEX-BLGm.

Constructs pGEX-mMUP and pGEX-BLGm were then used to produce recombinant GST fusion proteins in E. coli DH5α, and the GST fragments removed by protease treatment (PreScission Protease, Amersham Bioscience) to generate N-terminally myc-tagged MUP (mMUP—FIG. 8) and C-terminally myc-tagged BLG (BLGm—FIG. 9) lipocalin reporter proteins respectively. Purification of recombinant protein was achieved via affinity chromatography following the manufacturers recommended protocols (Amersham Bioscience).

Both the GST fusion precursors and the cleaved myc-tagged protein products were recognised on western blots (FIG. 10) using horseradish peroxidase (HRP) directly conjugated to an anti-myc antibody (9E10, Roche) and ECL chemiluminescent detection kit (Amersham Bioscience).

### EXAMPLE 7

### In Vivo Excretion of MUP and BLG Epitope Tagged Lipocalin Reporter Proteins

In order to demonstrate the excretion of epitope-tagged MUP and BLG reporter proteins, recombinant epitope-tagged in MUP lipocalin protein was injected i.v. into male CD1 mice (3 doses, 2.5 mg/kg, 5 mg/kg and 10 mg/kg with 3 mice per group, via the tail vein). A control group were also injected with the vehicle solution (isotonic sterile saline). After injection, urine samples were collected from mice, by scruffing, at approximately 30 minute time intervals over a 6 h period. Mice were sacrificed after 24 hours and urine and serum samples taken.

Urine was analysed by SDS PAGE, followed by western transfer to nitrocellulose membrane (Hybond ECL, Amersham Bioscience) and probed with HRP-conjugated anti-myc antibody (9E10, Roche) and detected with the ECL detection kit (Amersham Bioscience).

The results of this analysis are shown in FIG. 11. From this, it can be seen that the majority of MUP protein was detected in the first two or three samples i.e. within 2 h post injection. Urine samples collected at later time points and serum taken from animals after 24 h did not contain detectable MUP reporter protein. These data clearly demonstrate that exogenous mMUP in the bloodstream of mice is eliminated rapidly and efficiently in the urine.

Western blot analysis was repeated on all samples after three weeks to determine the stability of recombinant protein in mouse urine upon storage at −20° C. The results were similar to those initially obtained (data not shown), showing no appreciable decrease in sensitivity, demonstrating that mMUP protein is able to withstand long term freezer storage and thawing.

In order to demonstrate the application of lipocalin reporter proteins containing a large epitope tag (GST), tail vein injections were conducted subsequently with recombinant myc-tagged lipocalin-GST fusion proteins (GST-BLGm and GST-mMUP). Each protein was injected at a dose of 5 mg/kg. Samples were fractionated by SDS PAGE and analysed by western blotting. Blots were probed using an anti-GST antibody (Sigma), HRP-conjugated anti-rabbit secondary antibody (Jackson ImmunoResearch) and ECL detection kit (Amersham Bioscience). Urine samples collected early and late after IV injection and plasma from a terminal bleed were included in the analysis. From FIG. 12, it can be seen that GST-BLGm and GST-mMUP proteins are detected in urine samples throughout the sampling period and also in plasma taken from the animal after 24 hours.

The difference in excretion profiles between GST-mMUP fusion protein (45 kDa mol. weight) and mMUP (˜18 kDa mol. weight) could reflect a difference in the physiological processing of the former (e.g. reabsorption via the kidney into the plasma) or less efficient excretion. A choice of non-invasive reporter molecule whose excretion characteristics differ in such a manner could prove useful, depending on whether a persistent readout or a more rapidly decaying, and thus responsive, signal are required.

### EXAMPLE 8

### Epitope Tagging of Lipocalin Reporter Protein

MUP and BLG lipocalin reporter proteins have been successfully tagged with N- and C-terminal tags (above data for GST and c-myc tags). Internal loop positions within the MUP protein have also been used to introduce the peptide epitope sequences. Several potential positions for the introduction of epitope tags were chosen, from the MUP protein structure (FIG. 15), as being in external loops. The initial position chosen to introduce a tag corresponded to a site within the EF loop of BLG protein that had previously been used to introduce a kinase recognition site. This had utilised a ClaI restriction site in the BLG gene, however there is no corresponding restriction site in the MUP gene. Consequently, the Mup cDNA sequence was modified by the introduction of a) an AvrII-ApaI-SbfI linker fragment into the sequence coding for EF loop region and b) a SpeI-EcoRI-NsiI linker fragment at the 3′end of the coding sequence. The particular restriction site combinations were chosen since they would generate compatible overhanging ends, for the insertion of adapter oligonucleotides containing epitope sequences. The MU 5′-coding region from position 10 to 300, together with an additional GATGCGGTACCACCATGGTGTCTAGACTGCAG 5′-sequence (containing a Kozak signal, start codon and NcoI-KpnI-XbaI-PstI linker) and an additional CCTAGGC sequence (containing an AvrII restriction site) was generated by PCR. The corresponding MUP 3′-region from position 301 to 540, together with an additional TGCCTAGGGCCCTGCAGGGTA 5′-sequence (containing an AvrII-ApaI-SbfI linker) and ACTAGTGAATTCATGCATTGAGCTAGCCATC 3′sequence (containing an SpeI-EcoRI-NsiI-NheI linker and stop codon was generated by PCR. Ligation of these two fragments, at the common AvaII site generated the required modified MUP coding sequence, on a NcoI-NheI fragment.

Restriction digest with either AvrII/SbfI (internal EF loop) or SpeI/NsiI (C-terminus) results in an identical pattern of overhanging ends, to which double stranded oligonucleotide linkers, of the general form:

CTAG N (NNN)ₓ N TGCA

N (NNN)ₓ
   where x is a multiple of 3, that contain an epitope tag, can anneal.

MUP lipocalin reporter proteins have also been produced, in which the epitope has been introduced into the FG loop position. This has been accomplished by the insertion of a HindIII-BamHI-EcoRI linker fragment into the MUP coding sequence at the FG loop position. This has allowed the insertion of adapter oligonucleotides containing epitope sequences into the HindIII/EcoRI sites. The MUP coding sequence, from position 1 to 348, together with an additional GGTACCACC 5′-sequence (containing a KpnI restriction site and Kozak sequence) and an additional AAGCTTGGAACCGGATCC 3′-sequence (containing HindIII-BamHI sites) was generated by PCR, as was the corresponding MUP coding sequence from position 349 to 540, together with an additional GGATCCTCTTCAGAATTC 5′-sequence (containing BamHI and EcoRI restriction sites) and an additional GAGCAGAAACTCATCTCTGAAGAGGATCTGTGAGCTAGC 3′-sequence (containing the c-myc GluGlnLysLeuleSeTGluGluAspLeu epitope tag, stop codon and NheI restriction site). Ligation of the two fragments, at the BamHI site generated the modified MUP coding sequence, on a NcoI-NheI fragment.

Restriction digest with HindIII/EcoRI results in overhanging ends, to which double stranded oligonucleotide linkers, of the general form:

AGCT T (NNN)ₓ G

A (NNN)ₓ C TTAA
   where x is a multiple of 3, that contain an epitope tag, can anneal.

Epitopes that have been inserted into the FG loop, by this method, include:

| | |
|---|---|
| Haemaglutinin | (YPYDVPDYA) |
| Clone100 | (NVRFSTIVRRRA) |
| rab11a | (KQMSDRRENDMSPS) |
| DOB | (SGNEVSRAVLLPQSC) |
| SG11 | (SSLSYTNPAVAATSANL) |
| erbB4 | (RSTLQHPDYLQEYST) |
| ARF | (VSTLLRWERFPGHRQA) |
| RYK | (KFQQLVQCLTEFHAALGAYV) |
| WILPEP1 | (QEQCQEVWRKRVISAFLKSP) |
| HAF10 | (RLSDKTGPVAQEKS) |

MUP coding sequences, containing these epitope tag sequences, were expressed in E. coli as GST fusion precursor proteins, and cleaved tagged MUP proteins, using the pGEX expression system (Amersham Biosciences).

FG loop modified MUP coding sequence was cloned into NcoI-NotI cut pGEX6P vector to generate pGSLM, that contains the MUP coding region downstream of the GST coding sequence and Precissionase cleavage site.

Individual epitope tags were introduced by HindIII/EcoRI digestion and annealing of epitope containing oligonucleotide linkers.

E. coli strain TOP10 (Invitrogen) was transformed with the pGSLM-tag construct, using the manufacturers standard protocols.

The resultant transformed bacterial strains were grown in shaking flask culture to an OD₆₀₀ of 0.5-0.6. Once the optimal turbidity was attained a small sample was removed as a control and IPTG added to the remaining culture to a final concentration of 0.5 mM. Both the control sample (uninduced) and the induced cultures were grown for a further 2-3 hours. After the final growth step 0.25 ml and 0.5 ml of uninduced and induced culture respectively was spun down and resuspended in 100 ul 6×GLB and 5-10 ul of each run on NuPAGE gels (Invitrogen) to ascertain whether induction had taken place and the fusion product was the correct size.

The remaining induced culture (3.2 L total for large preps) was spun down, lysed and cell debris removed by centrifugation. GST fusion proteins from cleared lysate were allowed to bind to Glutathione-Agarose beads (SIGMA) for 0.5-1 hour at +4° C. The protein/bead slurry was poured onto a gravity flow column and the resultant gel bed washed thoroughly with lysis buffer to remove bacterial proteins. Fusion proteins were then eluted from the gel bed with excess Glutathione (10 mM in 50 mM Tris pH8.0). Samples were checked via SDS-PAGE and Immunodetection before proceeding to cleave and purify the tagged MUP protein from the GST fusion. The purified eluate was dialysed in cleavage buffer (4×3 hours) and then incubated for 16 hours with at least 60 units of Precissionase at +4° C. The digested protein was then added to a gravity flow column containing fresh Glutathione-Agarose beads which bound the GST and Precissionase allowing the elution of the cleaned, digested tagged MUP protein. The eluate was re-added twice to ensure complete removal of contaminating proteins and then concentrated using Centricon-P20 columns (Millipore) to give the final protein solution.

Extracts from induced and uninduced cells were analysed by western blotting for the presence of the relevant tagged MUP protein, using an epitope-specific monoclonal antibody. Some representative results are shown in FIG. 14.

### EXAMPLE 9

### In Vivo Expression and Secretion of Lipocalin Reporter Proteins

It is possible that modifying the protein sequence, by the introduction of epitopes, would affect protein folding or secretion. In order to examine this, we have expressed the modified MUP proteins in murine Hepa1-6 hepatoma cells and in human A2780 ovarian carcinoma cells.

MUP lipocalin reporter sequences, containing internal modifications at protein loop positions, were cloned into the pSecTag2 vector (Invitrogen). This vector contains a murine Ig Kappa signal peptide, a 3′-c-myc and His tag, and is designed to express tagged secreted proteins in mammalian cells.

In this way, 4 MUP reporter constructs, coding for proteins that contain epitope tag modifications at either the N-terminus, the C-terminus or at the internal FG loop position, were created (FIG. 15).

The DNA constructs were transfected into both murine Hepa1-6 hepatoma cells and human A2780 ovarian carcinoma cells, using Fugene transfection reagent (Invitrogen). After 72 h, medium was collected and analysed for the presence of secreted protein by western blotting. A typical blot is shown in FIG. 16.

The results demonstrate that MUP lipocalin reporter proteins, containing multiple modifications, are properly folded and secreted from mammalian cells.

### EXAMPLE 10

### Enzyme Detection of Lipocalin Protein

To demonstrate the detection of a lipocalin reporter by means of an epitope tag that contains enzymic activity, we have examined the GST enzymic activity of the GST-tagged MUP lipocalin reporter protein.

Mouse urine, that bad previously been spiked with GST-mMUP protein (100 μg/ml) was analysed for GST enzymic activity using a colorimetric assay (GST-Tag Kit, Novagen). The assay was performed according to the manufacturers recommended protocol, using a Hitachi-U3010 spectrophotometer and Hitachi UV Solutions Version 1.2 software. Absorbance was measured at 340 nm. Readings were taken every 30 seconds for 300 seconds

The results show that GST-mMUP lipocalin reporter protein can be efficiently detected in mouse urine by means of GST enzymic activity (FIG. 17). The activity of the GST-mMUP protein, in both urine and PBS, is similar to that of GST protein itself.

### EXAMPLE 11

### Expression of Epitope Tagged Lipocalin Reporter Proteins in Transgenic Animals

Transgenic animals are generated using one of several standard methods including pronuclear injection (Gordon and Ruddle, Science 214, 1244-1246 (1981)), blastocyst injection of transfected cells (Smithies et al., Nature 317, 230-234 (1985)) or using viral vectors (Lois et al., Science 295, 868-872 (2002); Pfeifer et al., Proc. Natl. Acad. Sci. USA 99, 2140-2145 (2002)). The transgene comprises DNA fragments including a promoter sequence driving an open reading frame encoding a tagged-lipocalin.

For example transgenes contain the mouse Cyp1a1 promoter sequence driving expression of myc epitope tagged MUP or BLG reporters, as follows:

pXC3′mycMUP. A 2.4 Kb fragment encompassing the murine Cyp1a1 promoter was derived by PCR from murine genomic DNA. This was cloned into the vector pXen5s (CXR Biosciences) as a SpeI/XhoI fragment to yield the vector pXen5Cyp. The Cyp1a promoter was subsequently moved from pXen5Cyp into the vector pXen4.3′mycMUP (CXR Biosiences) as an SstII/XhoI fragment replacing the CMV promoter contained in this vector. The resultant vector pXC3′mycMUP contains a C-terminally tagged MUP reporter running under the control of the murine Cyp1a1 promoter.

pXC3′mycBLG. The BLG reporter was amplified from the vector pBLacD (Roslin Institute) by PCR, adding flanking XhoI and KpnI sites and inserting a C-terminal Myc epitope tag. This fragment was digested XhoI/KpnI and used to replace the MUP reporter in XhoI/KpnI digested pXC3′mycMUP vector. The resultant vector pXC3′mycBLG contains a C-terminally tagged BLG reporter running under the control of the murine Cyp1a1 promoter.

Positive transgenic animals are identified by analysis of DNA (Whitelaw et al., Transgenic Res. 1, 3-13 (1991)) and bred to generate transgenic lines. Transgenic animals are exposed to stress, for example by drug administration, and blood and urine collected over time. Samples collected pre- and post-insult are analysed for the presence of the tagged-lipocalin by standard methods, including Western blot and ELISA. Depending on the specific insult or inducing agent an increase or decrease in reporter activity are detected.

Transgenes may also be refined to allow expression in specific cells, for example through the DNA recombination based strategies (Fiering et al., Proc. Natl. Acad. Sci. USA 90, 8469-8473 (1993); Gu et al., Cell 73, 1155-1164 (1993)).

Alternatively DNA promoter-reporter constructs are introduced into somatic cells of an animal. This could be achieved through the use of adenovirus (Lai et al., DNA Cell Biol. 21, 895-913 (2002), other viral vector methods (Logan et al., Curr. Opin. Bioetcnol. 13, 429-436 (2002)) or by non-viral methods including the direct introduction of naked DNA (Niidome and Huang, Gene Ther. 9, 1647-1652 (2002).

1-21. (canceled)

22. A method of detecting a gene activation event in a cell in vitro or in vivo, the method comprising assaying a host cell stably transfected with a nucleic acid construct comprising a nucleic acid sequence encoding a member of the lipocalin protein family, or a transgenic non-human animal whose cells express such a construct, in which the cell or animal is subjected to a gene activation event that is signaled by expression of a peptide tagged lipocalin reporter gene.

23. The method of claim 22, wherein the lipocalin protein is heterologous to the cell in which it is expressed.

24. The method of claim 22, wherein the lipocalin protein is coded for by a nucleic acid construct comprising (i) a nucleic acid sequence encoding a member of the lipocalin protein family, and (ii) a nucleic acid sequence encoding a peptide sequence of from 5 to 250 amino acid residues.

25. The method of claim 22, wherein the lipocalin is selected from the group consisting of: ovine betalactoglobulin (BLG) (accession No. X12817), murine major urinary protein (MUF) (accession No. NM 031188) and rat α-2-urinary globulin (α-2u) (accession number M27434).

26. The method of claim 24, wherein the peptide sequence is an epitope.

27. The method of claim 26, wherein the epitope is selected from the group consisting of EQKLISEEDL, GKPIPNPLLGLDST, YPYDVPDYA, NVRFSTTVRRRA, KQMSDRRENDMSPS, SGNEVSRAVLLPQSC, SSLSYTNPAVAATSANL, RSTLQHPDYLQEYST, VSTLLRWERFPGHRQA, KFQQLVQCLTEFHAALGAYV, QEQCQEVWRKRVISAFLKSP, and RLSDKTGPVAQEKS.

28. The method of claim 23, wherein the construct additionally comprises a promoter element upstream of the nucleic acid sequence comprising (i) a nucleic acid sequence encoding a member of the lipocalin protein family, and (ii) and nucleic acid sequence encoding a peptide sequence of from 5 to 250 amino acid residues.

29. The method of claim 7, wherein the promoter element is selected from one of the groups consisting of:
c-myc, p21/WAF-1, MDM2, Gadd45, FasL, GAHSP40, TRAIL-R2/DR5, BTG2/PC3; MnSOD, CuZnSOD, IKB, ATF4, xanthine oxidase, COX2, INOS, Ets-2, FasL/CD95L, YGCS, ORP150;
Lrg-21, SOCS-2, SOCS-3, PAI-1, GBP28/adiponectin, α-1 acid glycoprotein, metallothioneine I, metallothioneine II, ATF3, IGFbp-3, VDGF, HIF1α;
Gadd 34, GAHSP40, TRAIL-R2/DR5, c-fos, CHOP/Gadd153, APAF-1, Gadd45, BTG2/PC3, Peg3/Pw1, Siahla, S29 ribosomal protein, FasUCD95L, tissue tranglutaminase, GRP78, Nur77/NGFI-B, CyclophilinD, p73, Bak;
a promoter from a xenobiotic metabolizing cytochrome p450 enzyme from the 2A, 2B, 2C, 2D, 2E, 2S, 3A, 4A and 48 gene families; and
a synthetic promoter sequence comprised of a minimal eukaryote consensus promoter operatively linked to one or more response elements selected from the group consisting of the aryl hydrocarbon (Ah)/Ah nuclear translocator (ARNT) receptor response element, the antioxidant response element (ARE), the xenobiotic response element (XRE).

30. The method of claim 22, wherein the nucleic acid construct comprises a stress inducible promoter operatively isolated from a nucleic acid sequence encoding a member of the lipocalin protein family by a nucleotide sequence flanked by nucleic acid sequences recognized by a site specific recombinase, or by insertion such that it is inverted with respect to the transcription unit encoding a member of the lipocalin protein family, in which the construct additionally comprises a nucleic acid sequence comprising a tissue specific promoter operatively linked to a gene encoding the coding sequence for the site specific recombinase.

31. The method of claim 30, wherein the site-specific recombinase sequences are two loxP sites of bacteriophage P1.

32. The method of claim 22, wherein the gene activation event is induction of toxicological stress, metabolic changes, or disease, including a disease that is the result of viral, bacterial, fungal or parasitic infection.

33. A method of screening for, or monitoring of toxicologically induced stress in a cell or a cell line or a non-human animal, comprising the use of a cell, cell line or non human animal which has been transfected with or carries a nucleic acid construct as defined in claim 24.

34. A method for screening and characterizing viral, bacterial, fungal, and parasitic infection comprising the use of a cell, cell line or non human animal which has been transfected with or carries a nucleic acid construct as defined in claim 24.

35. A method for screening for cancer, inflammatory disease, cardiovascular disease, metabolic disease, neurological disease and disease with a genetic basis comprising the use of a cell, cell line or non human animal which has been transfected with or carries a nucleic acid construct as defined in claim 24.


SEQUENCE LISTING (41 sequences)

SEQ ID NO: 1 - protein, 10 aa, Homo sapiens
EQKLISEEDL

SEQ ID NO: 2 - protein, 14 aa, Simian virus
GKPIPNPLLGLDST

SEQ ID NO: 3 - protein, 9 aa, Unknown
  Description of Unknown Sequence Haemaglutinin epitope from unknown organism
YPYDVPDYA

SEQ ID NO: 4 - protein, 12 aa, Unknown
  Description of Unknown Sequence Clone100 epitope from unknown organism
NVRFSTIVRRRA

SEQ ID NO: 5 - protein, 14 aa, Unknown
  Description of Unknown Sequence Rab11a epitope from unknown organism
KQMSDRRENDMSPS

SEQ ID NO: 6 - protein, 15 aa, Unknown
  Description of Unknown Sequence DOB epitope from unknown organism
SGNEVSRAVLLPQSC

SEQ ID NO: 7 - protein, 17 aa, Unknown
  Description of Unknown Sequence SG11 epitope from unknown organism
SSLSYTNPAVAATSANL

SEQ ID NO: 8 - protein, 15 aa, Unknown
  Description of Unknown Sequence ErbB4 epitope from unknown organism
RSTLQHPDYLQEYST

SEQ ID NO: 9 - protein, 16 aa, Unknown
  Description of Unknown Sequence ARF epitope from unknown organism
VSTLLRWERFPGHRQA

SEQ ID NO: 10 - protein, 20 aa, Unknown
  Description of Unknown Sequence RYK epitope from unknown organism
KFQQLVQCLTEFHAALGAYV

SEQ ID NO: 11 - protein, 20 aa, Unknown
  Description of Unknown Sequence WILPEP1 epitope from unknown organism
QEQCQEVWRKRVISAFLKSP

SEQ ID NO: 12 - protein, 14 aa, Unknown
  Description of Unknown Sequence HAF10 epitope from unknown organism
RLSDKTGPVAQEKS

SEQ ID NO: 13 - DNA, 543 bp, Mus musculus
  CDS (1)..(540)
atgaagatgctgctgctgctgtgtttgggactgaccctagtctgtgtccatgcagaagaagctagttctacgggaaggaactttaatgtagaaaagattaatggggaatggcatactattatcctggcctctgacaaaagagaaaagatagaagataatggcaactttagactttttctggagcaaatccatgtcttggagaaatccttagttcttaaattccatactgtaagagatgaagagtgctcggaattatctatggttgctgacaaaacagaaaaggctggtgaatattctgtgacgtatgatggattcaatacatttactatacctaagacagactatgataactttcttatggctcatctcattaacgaaaaggatggggaaaccttccagctgatggggctctatggccgagaaccagatttgagttcagacatcaaggaaaggtttgcacaactatgtgagaagcatggaatccttagagaaaatatcattgacctatccaatgccaatcgctgcctccaggcccgagaatga

SEQ ID NO: 14 - protein, 180 aa, Mus musculus
MKMLLLLCLGLTLVCVHAEEASSTGRNFNVEKINGEWHTIILASDKREKIEDNGNFRLFLEQIHVLEKSLVLKFHTVRDEECSELSMVADKTEKAGEYSVTYDGFNTFTIPKTDYDNFLMAHLINEKDGETFQLMGLYGREPDLSSDIKERFAQLCEKHGILRENIIDLSNANRCLQARE

SEQ ID NO: 15 - DNA, 540 bp, Artificial Sequence
  Description of Artificial Sequence Synthetic recombinant mMUP reporter molecule
gggcccctgggatccatggagcagaaactcatctctgaagaggatctgaccatggaagctagttctacgggaaggaactttaatgtagaaaagattaatggggaatggcatactattatcctggcctctgacaaaagagaaaagatagaagataatggcaactttagactttttctggagcaaatccatgtcttggagaaatccttagttcttaaattccatactgtaagagatgaagagtgctcggaattatctatggttgctgacaaaacagaaaaggctggtgaatattctgtgacgtatgatggattcaatacatttactatacctaagacagactatgataactttcttatggctcatctcattaacgaaaaggatggggaaaccttccagctgatggggctctatggccgagaaccagatttgagttcagacatcaaggaaaggtttgcacaactatgtgagaagcatggaatccttagagaaaatatcattgacctatccaatgccaatcgctgcctccaggcccgagaatga

SEQ ID NO: 16 - protein, 179 aa, Artificial Sequence
  Description of Artificial Sequence Synthetic recombinant mMUP reporter molecule
GPLGSMEQKLISEEDLTMEASSTGRNFNVEKINGEWHTIILASDKREKIEDNGNFRLFLEQIHVLEKSLVLKFHTVRDEECSELSMVADKTEKAGEYSVTYDGFNTFTIPKTDYDNFLMAHLINEKDGETFQLMGLYGREPDLSSDIKERFAQLCEKHGILRENIIDLSNANRCLQARE

SEQ ID NO: 17 - DNA, 540 bp, Artificial Sequence
  Description of Artificial Sequence Synthetic recombinant BLGm reporter molecule
gggcccctgggatccatggccatcatcgtcacccagaccatgaaaggcctggacatccagaaggtggcggggacttggcactccttggctatggcggccagcgacatctccctgctggatgcccagagtgcccccctgagagtgtacgtggaggagctgaagcccacccccgagggcaacctggagatcctgctgcagaaatgggagaacggcgagtgtgctcagaagaagattattgcagaaaaaaccaagatccctgcggtgttcaagatcgatgccttgaatgagaacaaagtccttgtgctggacaccgactacaaaaagtacctgctcttctgcatggaaaacagtgctgagcccgagcaaagcctggcctgccagtgcctggtcaggaccccggaggtggacaacgaggccctggagaaattcgacaaagccctcaaggccctgcccatgcacatccggcttgccttcaacccgacccagctggaggggcagtgccacgtcgagcagaaactcatctctgaagaggatctgtag

SEQ ID NO: 18 - protein, 179 aa, Artificial Sequence
  Description of Artificial Sequence Synthetic recombinant BLGm reporter molecule
GPLGSMAIIVTQTMKGLDIQKVAGTWHSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHVEQKLISEEDL

SEQ ID NO: 19 - protein, 214 aa, Artificial Sequence
  Description of Artificial Sequence Synthetic modified MUP protein produced from the pSecTag vector
METDTLLLWVLLLWVPGSTGDAAQPARRARRTKLGTELGSMEQKLISEEDLTMEASSTGRNFNVEKINGEWHTIILASDKREKIEDNGNFRLFLEQIHVLEKSLVLKFHTVRDEECSELSMVADKTEKAGEYSVTYDGFNTFTIPKTDYDNFLMAHLINEKDGETFQLMGLYGREPDLSSDIKERFAQLCEKHGILRENIIDLSNANRCLQARE

SEQ ID NO: 20 - protein, 243 aa, Artificial Sequence
  Description of Artificial Sequence Synthetic modified MUP protein produced from the pSecTag vector
METDTLLLWVLLLWVPGSTGDAAQPAKMLLLLCLGLTLVCVHAEEASSTGRNFNVEKINGEWHTIILASDKREKIEDNGNFRLFLEQIHVLEKSLVLKFHTVRDEECSELSMVADKTEKAGEYSVTYDGFNTFTIPKTDYDNFLMAHLINEKDGETFQLMGLYGREPDLSSDIKERFAQLCEKHGILRENIIDLSNANRCLQAREEQKLISEEDLAAARGGPEQKLISEEDLNSAVDHHHHHH

SEQ ID NO: 21 - protein, 253 aa, Artificial Sequence
  Description of Artificial Sequence Synthetic modified MUP protein produced from the pSecTag vector
METDTLLLWVLLLWVPGSTGDAAQPAKMLLLLCLGLTLVCVHAEEASSTGRNFNVEKINGEWHTIILASDKREKIEDNGNFRLFLEQIHVLEKSLVLKFHTVRDEECSELSMVADKTEKAGEYSVTYDGFNTFTIPKTDYDKLGTGSSSEFNFLMAHLINEKDGETFQLMGLYGREPDLSSDIKERFAQLCEKHGILRENIIDLSNANRCLQAREEQKLISEEDLAAARGGPEQKLISEEDLNSAVDHHHHHH

SEQ ID NO: 22 - protein, 259 aa, Artificial Sequence
  Description of Artificial Sequence Synthetic modified MUP protein produced from the pSecTag vector
METDTLLLWVLLLWVPGSTGDAAQPAKMLLLLCLGLTLVCVHAEEASSTGRNFNVEKINGEWHTIILASDKREKIEDNGNFRLFLEQIHVLEKSLVLKFHTVRDEECSELSMVADKTEKAGEYSVTYDGFNTFTIPKTDYDKLNVRFSTIVRRRAEFNFLMAHLINEKDGETFQLMGLYGREPDLSSDIKERFAQLCEKHGILRENIIDLSNANRCLQAREEQKLISEEDLAAARGGPEQKLISEEDLNSAVDHHHHHH

SEQ ID NO: 23 - DNA, 7379 bp, Ovis aries
gtgctcagcaacacacccagcaccagcattcccgctgctcctgaggtctgcaggcagctcgctgtagcctgagcggtgtggagggaagtgtcctgggagatttaaaatgtgagaggcgggaggtgggaggttgggccctgtgggcctgcccatcccacgtgcctgcattagccccagtgctgctcagccgtgcccccgccgcaggggtcaggtcactttcccgtcctggggttattatgactcttgtcattgccattgccatttttgctaccctaactgggcagcaggtgcttgcagagccctcgataccgaccaggtcctccctcggagctcgacctgaaccccatgtcacccttgccccagcctgcagagggtgggtgactgcagagatcccttcacccaaggccacggtcacatggtttggaggagctggtgcccaaggcagaggccaccctccaggacacacctgtccccagtgctggctctgacctgtccttgtctaagaggctgaccccggaagtgttcctggcactggcagccagcctggacccagagtccagacacccacctgtgcccccgcttctggggtctaccaggaaccgtctaggcccagagggggacttcctgcttggccttggatggaagaaggcctcctattgtcctcgtagaggaagccaccccggggcctgaggatgagccaagtgggattccgggaaccgcgtggctgggggcccagcccgggctggctggcctgcatgcgcctcctgtataaggccccaagcctgcctgtctcagccctccactccctgcagagctcagaagcacgaccccagctgcagccatgaagtgcctcctgcttgccctgggcctggccctcgcctgtggcgtccaggccatcatcgtcacccagaccatgaaaggcctggacatccagaaggttcgagggttggccgggtgggtgagttgcagggcgggcaggggagctgggcctcagagagccaagagaggctgtgacgttgggttcccatcagtcagctagggccacctgacaaatccccgctggggcagcttcaaccaggcgttcactgtcttgcattctggaggctggaagcccaagatccaggtgttggcagggctggcttctcctgcggccgctctctggggagcagacggccgtcttctccagtcctctgcgcgccctgatttcctcttcctgtgaggccaccaggcctgctggaaacacgcctgcctgcgcagcttcacacgacctttgtcatctctttaaaggccatgtctccagagtcatgtgttgaagttctgggggttagtgggacacagttcagcccctaaaagagtctctctgcccctcaaattttccccacctccagccatgtctccccaagatccaaatgttgctacatgtgggggggctcatctgggtccctctttgggttcagtgtgagtctggggagagcattccccagggtgcagagttggggggagtatctcagggctgcccaggccggggtgggacagagagcccactgtggggctgggggccccttcccacccccagagtgcaactcaaggtccctctccaggtggcggggacttggcactccttggctatggcggccagcgacatctccctgctggatgcccagagtgcccccctgagagtgtacgtggaggagctgaagcccacccccgagggcaacctggagatcctgctgcagaaatggtgggcgtctctccccaacatggaacccccactccccagggctgtggaccccccggggggtggggtgcaggagggaccagggccccagggctggggaagagggctcagagtttactggtacccggcgctccacccaaggctgcccacccagggctttttttttttttaaacttttattaatttgatgcttcagaacatcatcaaacaaatgaacataaaacattcatttttgtttacttggaaggggagataaaatcctctgaagtggaaatgcatagcaaagatacatacaatgaggcaggtattctgaattccctgttagtctgaggattacaagtgtatttgagcaacagagagacattttcatcatttctagtctgaacacctcagtatctaaaatgaacaagaagtcctggaaacgaagcagtgtggggataggcccgtgtgaaggctgctgggaggcagcagacctgggtcttcgggctcaagcagttcccgctaccagccctgtccacctcagacgggggtcagggtgcaggagagagctggatgggtgtgggggcagagatggggacctgaaccccagggctgccttttgggggtgcctgtggtcaaggctctccctgaccttttctctctggcttcatctgacttctcctggcccatccacccggtcccctgtggcctgaggtgacagtgagtgcgccgaggctagttggccagctggctcctatgcccatgccacccccctccagccctcctgggccagcttctgcccctggccctcagttcatcctgatgaaaatggtccatgccaatggctcagaaagcagctgtctttcagggagaacggcgagtgtgctcagaagaagattattgcagaaaaaaccaagatccctgcggtgttcaagatcgatggtgagtccgggtccctgggggacacccaccacccccgcccccggggactgtggacaggttcagggggctggcgtcgggccctgggatgctaagggactggtggtgatgaagacactgccttgacacctgcttcacttgcctcccctgccacctgcccggggccttggggcggtggccatgggcaggtcccggctggcgggctaacccaccagggtgacacccgagctctctttgctggggggcgggcggtgctctgggccctcaggctgagctcaggaggtacctgtgccctcccaggggtaaccgagagccgttgcccactccaggggcccaggtgccccacgaccccagcccgctccacagctccttcatctcctggagacaaactctgtccgccctcgctcattcacttgttcgtcctaaatccgagatgataaagcttcgagggggggttggggttccatcagggctgcccttccgccgggcagcctgggccacatctgcccttggccccctcaggactcactctgactggaggccctgcactgactgacgccagggtgcccagcccagggtctctggcgccatccagctgcactgggtttgggtgctggtcctgcccccaagctgcccggacaccacaggcagccggggctgcccactggcctcggtcagggtgagccccagctgcccccgctcagggcttgccccgacaatgaccccatcctcaggacgcaccccccttcccttgctgggcagtgtccagccccacccgagatcgggggaagccctatttcttgacaactccagtccctgggggagggggcctcagactgagtggtgagtgttcccaagtccaggaggtggtggagggtcctggcggatccagagttgacagtgagggcttcctgggccccatgcgcctggcagtggcagcagggaagaggaagcaccatttcaggggtgggggatgccagaggcgctccccaccccgtcttcgccgggtggtgaccccgggggagccccgctggtcgtggagggtgctgggggctgactagcaacccctccccccccgttggaactcacttttctcccgtcttgaccgcgtccagccttgaatgagaacaaagtccttgtgctggacaccgactacaaaaagtacctgctcttctgcatggaaaacagtgctgagcccgagcaaagcctggcctgccagtgcctgggtgggtgccaaccctggctgcccagggagaccagctgcgtggtccttgctgcaacagggggtggggggtgggagcttgatccccaggaggaggaggggtggggggtccctgagtcccgccaggagagagtggtcgcataccgggagccagtctgctgtgggcctgtgggtggctggggacgggggccagacacacaggccgggagacgggtgggctgcagaactgtgactggtgtgaccgtcgcgatggggccggtggtcactgaatctaacagcctttgttaccggggagtttcaattatttcccaaaataagaactcaggtacaaagccatctttcaactatcacatcctgaaaacaaatggcaggtgacattttctgtgccgtagcagtcccactgggcattttcagggcccctgtgccaggggggcgcgggcatcggcgagtggaggctcctggctgtgtcagccggcccagggggaggaagggacccggacagccagaggtggggggcaggctttccccctgtgacctgcagacccactgcactgccctgggaggaagggaggggaactaggccaagggggaagggcaggtgctctggagggcaagggcagacctgcagaccaccctggggagcagggactgacccccgtccctgccccatagtcaggaccccggaggtggacaacgaggccctggagaaattcgacaaagccctcaaggccctgcccatgcacatccggcttgccttcaacccgacccagctggagggtgagcacccaggccccgcccttccccagggcaggagccacccggccccgggacgacctcctcccatggtgacccccagctccccaggcctcccaggaggaaggggtggggtgcagcaccccgtgggggccccctccccaccccctgccaggcctctcttcccgaggtgtccagtcccatcctgacccccccatgactctccctcccccacagggcagtgccacgtctaggtgagcccctgccggtgcctctggggtaagctgcctgccctgccccacgtcctgggcacacacatggggtagggggtcttggtggggcctgggaccccacatcaggccctggggtcccccctgtgagaatggctggaagctggggtccctcctggcgactgcagagctggctggccgcgtgccactcttgtgggtgacctgtgtcctggcctcacacactgacctcctccagctccttccagcagagctaaggctaagtgagccagaatggtacctaaggggaggctagcggtccttctcccgaggaggggctgtcctggaaccaccagccatggagaggctggcaagggtctggcaggtgccccaggaatcacaggggggccccatgtccatttcagggcccgggagccttggactcctctggggacagacgacgtcaccaccgccccccccccatcagggggactagaagggaccaggactgcagtcacccttcctgggacccaggcccctccaggcccctcctggggctcctgctctgggcagcttctccttcaccaataaaggcataaacctgtgctctcccttctgagtctttgctggacgacgggcagggggtggagaagtggtggggagggagtctggctcagaggatgacagcggggctgggatccagggcgtctgcatcacagtcttgtgacaactgggggcccacacacatcactgcggctctttgaaactttcaggaaccagggagggactcggcagagacatctgccagttcacttggagtgttcagtcaacacccaaactcgacaaaggacagaaagtggaaaatggctgtctcttagtctaataaatattgatatgaaactcaagttgctcatggatcaatatgcctttatgatccagccagccactactgtcgtatcaactcatgtacccaaacgcactgatctgtctggctaatgatgagagattcccagtagagagctggcaagaggtcacagtgagaactgtctgcacacacagcagagtccaccagtcatcctaaggagatcagtcctggtgttcattggaggactgatgttgaagctgaaactccaatgctttggccacctgatgtgaagagctgactcatttgaaaagaccctgatgctgggaaagattgagggcaggaggagaaggggacgacagaggatgagatggttggatggcatcaccaacacaatggacatgggtttgggtggactccaggagttggtgatggacagggaggcctggcgtgctacggaagcggtttatggggtcacaaagactgagtgactgaactgagctgaactgaatggaaatgaggtatacagcaaagtggggattttttagataataagaatatacacataacatagtgtatactcatatttttatgcatacctgaatgctcagtcactcagtcgtatctgactctgtgacctatggaccgtagccttccaggtttcttctgtccacagaattctccaaggcaagaatactggagtgggtagccatttcctcctccaggggatcctcccgacccagggattgaaccggcatctcctgtattggcaggtggattctttaccactgtgccaccagggaagcccgtgttactctctatgtcccacttaattaccaaagctgctccaagaaaaagcccctgtgccctctgagcttcccggcctgcagagggtggtgggggtagactgtgacctgggaacaccctcccgcttcaggactcccgggccacgtgacccacagtcctgcagacagccgggtagctctgctcttcaaggctcattatctttaaaaaaaactgaggtctattttgtgacttcgctgccgtaacttctgaacatccagtgcgatggacaggacctcctccccaggcctcaggggcttcagggagccagccttcacctatgagtcaccagacactcgggggtggccccgccttcagggtgctcacagtcttcccatcgtcctgatcaaagagcaagaccaatgacttcttaggagcaagcagacacccacaggacactgaggttcaccagagctgagctgtccttttgaacctaaagacacacagctctcgaaggttttctctttaatctggatttaaggcctacttgcccctcaagagggaagacagtcctgcatgtccccaggacagccactcggtggcatccgaggccacttagtattatctgaccgcaccctggaattaatcggtccaaactggacaaaaaccttggtgggaagtttcatcccagaggcctcaaccatcctgctttgaccaccctgcatctttttttcttttatgtgtatgcatgtatatatatatatatatttttttttttttcattttttggctgtgctggctgttcgttgcagttcggtgcgcaggcttctctctagtttctctctagtcttctcttatcacagagcagtctctaga

SEQ ID NO: 24 - protein, 180 aa, Ovis aries
MKCLLLALGLALACGVQAIIVTQTMKGLDIQKVAGTWHSLAMAASDISLLDAQSAPLRVYVEELKPTPEGNLEILLQKWENGECAQKKIIAEKTKIPAVFKIDALNENKVLVLDTDYKKYLLFCMENSAEPEQSLACQCLVRTPEVDNEALEKFDKALKALPMHIRLAFNPTQLEGQCHV

SEQ ID NO: 25 - DNA, 925 bp, Mus musculus
ctgaacccagagagtatataagaacaagcaaaggggctggggagtggagtgtagccacgatcacaagaaagacgtggtcctgacagacagacaatcctattccctaccaaaatgaagatgctgctgctgctgtgtttgggactgaccctagtctgtgtccatgcagaagaagctagttctacgggaaggaactttaatgtagaaaagattaatggggaatggcatactattatcctggcctctgacaaaagagaaaagatagaagataatggcaactttagactttttctggagcaaatccatgtcttggagaattccttagttcttaaattccatactgtaagagatgaagagtgctcggaattatctatggttgctgacaaaacagaaaaggctggtgaatattctgtgacgtatgatggattcaatacatttactatacctaagacagactatgataactttcttatggctcatctcattaacgaaaaggatggggaaaccttccagctgatggggctctatggccgagaaccagatttgagttcagacatcaaggaaaggtttgcacaactatgtgagaagcatggaatccttagagaaaatatcattgacctatccaatgccaatcgctgcctccaggcccgagaatgaagaatggcctgagcctccagtgttgagtggagacttctcaccaggactccaccatcatcccttcctatccatacagcatccccagtataaattctgtgatctgcattccatcctgtctcactgagaagtccaattccagtctatccacatgttacctaggatacctcatcaagaatcaaagacttctttaaatttttctttgatatacccatgacaatttttcatgaatttcttcctcttcctgttcaataaatgattacccttgcactta

SEQ ID NO: 26 - protein, 180 aa, Mus musculus
MKMLLLLCLGLTLVCVHAEEASSTGRNFNVEKINGEWHTIILASDKREKIEDNGNFRLFLEQIHVLENSLVLKFHTVRDEECSELSMVADKTEKAGEYSVTYDGFNTFTIPKTDYDNFLMAHLINEKDGETFQLMGLYGREPDLSSDIKERFAQLCEKHGILRENIIDLSNANRCLQARE

SEQ ID NO: 27 - DNA, 813 bp, Rattus norvegicus
ctgctgctgctgtgtctgcgcctgacactggtctgtggccatgcagaagaagctagttccacaagagggaacctcgatgtggctaagctcaatggggattggttttctattgtcgtggcctctaacaaaagagaaaagatagaagagaatggcagcatgagagtttttatgcagcacatcgatgtcttggagaattccttaggcttcaagttccgtattaaggaaaatggagagtgcagggaactatacttggtttcctacaaaacgccagaggatggtgaatattttgttgagtatgacggagggaatacatttactatacttaagacagactactacatatacgtcatgtttcatctcattaatttcaagaacggggaaaccttccagctgatggtgctctacggcagaacaaaggatctgagttcagacatcaaggaaaagtttgcaaaactatgtgaggcgcatggaatcactagggacaatatcattgatctaaccaagactgatcgctgtctccaggcccgaggatgaagaaaggcctgagcctccagtgctgagtggagacttctcaccaggactctagcatcaccatttcctgtccatggagcatcctgagacaaattctgcgatctgatttccatcctctgtcacagaaaagtgcaatcctggtctctccagcatcttccctagttacccaggacaacacatcgagaattaaaagctttcttaaatttctcttggccccacccatgatcattccgcacaaatatcttgctcttgcagttcaataaatgattacccttgcacttt

SEQ ID NO: 28 - DNA, 735 bp, Artificial Sequence
  Description of Artificial Sequence Synthetic GST coding sequence derived from pGEX6p-1
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccggaattcccgggtcgactcgagcggccgcatcgtgactga

SEQ ID NO: 29 - DNA, 687 bp, Artificial Sequence
  Description of Artificial Sequence Synthetic GST coding sequence derived from pGEX6p-1
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctg

SEQ ID NO: 30 - protein, 229 aa, Artificial Sequence
  Description of Artificial Sequence Synthetic GST coding sequence derived from pGEX6p-1
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPL

SEQ ID NO: 31 - DNA, 24 bp, Artificial Sequence
  Description of Artificial Sequence Synthetic protease cleavage site
ctggaagttctgttccaggggccc

SEQ ID NO: 32 - protein, 8 aa, Artificial Sequence
  Description of Artificial Sequence Synthetic protease cleavage site
LEVLFQGP

SEQ ID NO: 33 - DNA, 32 bp, Artificial Sequence
  Description of Artificial Sequence Synthetic nucleotide sequence that contains a Kozak signal, start codon and NcoI-KpnI-XbaI-PstI linker
gatgcggtaccaccatggtgtctagactgcag

SEQ ID NO: 34 - DNA, 21 bp, Artificial Sequence
  Description of Artificial Sequence Synthetic nucleotide sequence that contains an AvrII-ApaI-SbfI linker
tgcctagggccctgcagggta

SEQ ID NO: 35 - DNA, 31 bp, Artificial Sequence
  Description of Artificial Sequence Synthetic nucleotide sequence that contains an SpeI-EcoRI-NsiI-NheI linker and stop codon
actagtgaattcatgcattgagctagccatc

SEQ ID NO: 36 - DNA, 13 bp, Artificial Sequence
  Description of Artificial Sequence Synthetic double stranded oligonucleotide linker
ctagnnnnntgca

SEQ ID NO: 37 - DNA, 18 bp, Artificial Sequence
  Description of Artificial Sequence Synthetic nucleotide sequence that contains HindIII-BamHI sites
aagcttggaaccggatcc

SEQ ID NO: 38 - DNA, 18 bp, Artificial Sequence
  Description of Artificial Sequence Synthetic nucleotide sequence that contains BamHI and EcoRI restriction sites
ggatcctcttcagaattc

SEQ ID NO: 39 - DNA, 39 bp, Artificial Sequence
  Description of Artificial Sequence Synthetic nucleotide sequence that contains c-myc epitope tag, stop codon and NheI restriction site
gagcagaaactcatctctgaagaggatctgtgagctagc

SEQ ID NO: 40 - DNA, 9 bp, Artificial Sequence
  Description of Artificial Sequence Synthetic double stranded oligonucleotide linker
agcttnnng

SEQ ID NO: 41 - DNA, 9 bp, Artificial Sequence
  Description of Artificial Sequence Synthetic double stranded oligonucleotide linker
aattcnnna